(19) United States
(12) Patent Application Publication
(10) Pub. No.: US 2017/0141320 A1
(43) Pub. Date: May 18, 2017

(54) PHOTOELECTRIC CONVERSION ELEMENT, AND IMAGE SENSOR, SOLAR CELL, SINGLE COLOR DETECTION SENSOR AND FLEXIBLE SENSOR EACH OF WHICH USES SAID PHOTOELECTRIC CONVERSION ELEMENT

(71) Applicant: TORAY INDUSTRIES, INC., Tokyo (JP)

(72) Inventors: Masaaki UMEHARA, Otsu-shi, Shiga (JP); Tsuyoshi TOMINAGA, Seoul (KR); Jinwoo KWON, Seoul (KR)

(73) Assignee: TORAY INDUSTRIES, INC., Tokyo (JP)

(21) Appl. No.: 15/317,856

(22) PCT Filed: Aug. 5, 2015
(86) PCT No.: PCT/JP2015/072229

§ 371 Date: Dec. 9, 2016

(30) Foreign Application Priority Data
Aug. 20, 2014 (JP) 2014-167325

(51) Int. Cl. H01L 51/00; C07D 307/79; C07C 15/38; H01L 51/42; H01L 27/30

(52) U.S. Cl. CPC H01L 51/0054; H01L 51/424; H01L 27/307; H01L 27/301; C07C 15/38; H01L 51/0058; C07D 307/79; H01L 51/0073; H01L 51/0055; H01L 51/0068; H01L 51/0053; H01L 51/0037; H01L 51/0035; C07C 2103/44; C07C 2103/52

(57) ABSTRACT

The present invention has the configuration described below for the purpose of providing a photoelectric conversion efficiency element which exhibits high charge mobility, while having high photoelectric conversion efficiency. A photoelectric conversion element which comprises at least one organic layer between a first electrode and a second electrode, and which is characterized in that the organic layer contains a first compound represented by general formula (1) and a second compound that has a maximum value of absorption coefficient of 5×10⁴ cm⁻¹ or more at a wavelength of 400-700 nm.

### TECHNICAL FIELD

The present invention relates to a photoelectric conversion element which can convert light into electric energy. More particularly, the present invention relates to a photoelectric conversion element which can be employed in the fields of solar cells, image sensors, and the like.

### BACKGROUND ART

The photoelectric conversion element capable of converting light into electric energy can be employed in solar cells, image sensors, and the like. In particular, there has widely been used an image sensor in which a current generated by incident light in a photoelectric conversion element is read out by CCD and CMOS circuits.

In an image sensor using a photoelectric conversion element, an inorganic substance has hitherto been employed as a material composing the photoelectric conversion film. However, since the inorganic substance has low selectivity of color (absorption of specific colors), it has been necessary that light of each of colors (red, green and blue) in incident light be selectively transmitted, and absorbed in a photoelectric conversion film. However, during imaging a fine object, use of the color filter may lead to an interference between the pitch of the object and that of an image element, thus generating an image which is different from an original image (Moire defects). For suppressing the defects, an optical lens etc. is needed, but there is a disadvantage that a color filter and an optical lens reduce efficiency for light utilization and an aperture ratio.

Meanwhile, growing demands for higher resolution of the image sensor create an opportunity of the progress of microfabrication of pixels. Accordingly, the size of pixels further decreases, and reduction in size of pixels leads to a decrease in quantity of light which reaches the photoelectric conversion element of each pixel, thus causing deterioration of sensitivity.

To solve these problems, a study has been made of a photoelectric conversion element using an organic compound. Since the organic compound can selectively absorb light in a specific wavelength region of light being incident according to a molecular structure, no color filter is required. Further, since the organic compound has a large absorption coefficient, efficiency for light utilization can be improved. There have been known, as a photoelectric conversion element using the organic compound, specifically, element structures in which a p-n junction structure and a bulk heterojunction structure are introduced into a photoelectric conversion film sandwiched between an anode and a cathode. For example, Patent Document 1 discloses an organic photoelectric material containing a compound having a thiophene-containing aromatic group in which an aromatic ring is fused.

### PRIOR ART DOCUMENTS

### Patent Documents

Patent Document 1: Japanese Patent Laid-open Publication No. 2014-17484

### SUMMARY OF THE INVENTION

### Problems to be Solved by the Invention

Although superiority of use of the photoelectric conversion element using an organic compound particularly in an image sensor can be confirmed, in principle, there are numerous technical problems for putting it into practical use.

For example, in Patent Document 1, a thiophene-based compound having a large absorption coefficient (hereinafter, referred to as a compound of Patent Document 1) is used. A photoelectric conversion element using the compound of Patent Document 1 exhibits relatively high photoelectric conversion efficiency, but further improvement of photoelectric conversion efficiency has been required.

Meanwhile, as organic compounds to be used in the photoelectric conversion element, many compounds having a large absorption coefficient (hereinafter, referred to as other light-absorbing compounds) are known in addition to the compound of Patent Document 1. However, with photoelectric conversion elements using these other light-absorbing compounds, sufficient photoelectric conversion efficiency is not obtained, and improvement of photoelectric conversion efficiency has been required.

Thus, an object of the present invention is to solve the problems of the prior art and to provide a photoelectric conversion element having higher photoelectric conversion efficiency.

### Solutions to the Problems

The inventors of the present application gave attention to the charge mobility of a photoelectric conversion element for solving the above-mentioned problems. Specifically, the reason why a photoelectric conversion element using the compound of Patent Document 1 exhibited relatively high photoelectric conversion efficiency, whereas photoelectric conversion elements using the other light-absorbing compounds did not exhibit sufficient photoelectric conversion efficiency was thought to be that the compound of Patent Document 1 had sufficient charge mobility, whereas the other light-absorbing compounds did not have sufficient charge mobility. Thus, the inventors of the present application tried to improve the charge mobility of the other light-absorbing compounds, but it was difficult to design and synthesize a molecule that improves charge mobility while maintaining a large absorption coefficient. Thus, the inventors of the present application conceived improvement of the photoelectric conversion efficiency of photoelectric conversion elements using the other light-absorbing compounds by combining the other light-absorbing compounds with a compound having sufficient charge mobility.

First, the inventors of the present application examined naphthacene as a compound having charge mobility. However, even when naphthacene was combined with the other light-absorbing compounds, high photoelectric conversion efficiency was not obtained. Thus, the inventors of the present application have further conducted studies repeatedly, and found that high photoelectric conversion efficiency is obtained by combining the other light-absorbing compounds with a fused ring aromatic compound having a specific structure. The present invention is as follows.

The present invention is directed to a photoelectric conversion element including a first electrode, a second electrode, and at least one organic layer existing therebetween, the organic layer containing a first compound, and a second compound in which the maximum value of an absorption coefficient at a wavelength of 400 to 700 nm is 5×10⁴ cm⁻¹ or more, the first compound being represented by the following general formula (1):

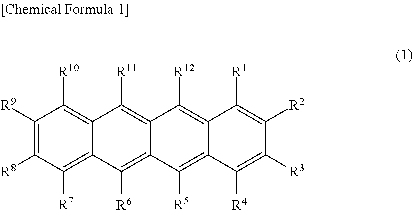

(in the general formula (1), R¹ to R¹² may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, a carbonyl group, a carboxyl group, an oxycarbonyl group, a carbamoyl group, an amino group, a nitro group, a cyano group, a silyl group and —P(═O)R¹³R¹⁴; R¹³ and R¹⁴ each represent an aryl group or a heteroaryl group; adjacent substituents may be linked together to form a ring structure; and

R⁵ and R¹² in the general formula (1) each represent a group represented by the following general formula (2) or (3):

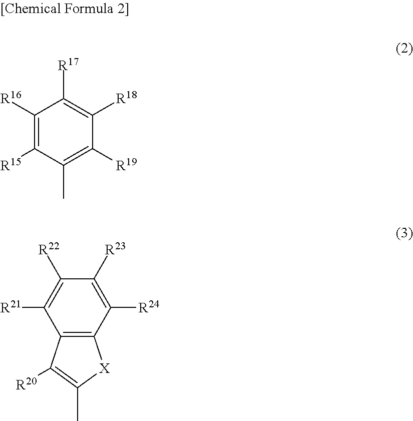

(in the general formula (2) or (3), R¹⁵ to R²⁴ may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, a carbonyl group, a carboxyl group, an oxycarbonyl group, a carbamoyl group, an amino group, a nitro group, a cyano group, a silyl group and —P(═O)R¹³R¹⁴; R¹³ and R¹⁴ each represent an aryl group or a heteroaryl group; R¹⁵ to R¹⁹ and R²¹ to R²⁴ may form a ring between adjacent substituents; X represents an oxygen atom, a sulfur atom or —NR²⁵; and R²⁵ is hydrogen, an alkyl group, a cycloalkyl group, a heterocyclic group, an aryl group or a heteroaryl group)).

### Effect of the Invention

According to the present invention, it is possible to provide a photoelectric conversion element having high photoelectric conversion efficiency.

### BRIEF DESCRIPTION OF THE DRAWINGS

FIG. 1 is a schematic sectional view showing an example of a photoelectric conversion element of the present invention.

FIG. 2 is a schematic sectional view showing an example of a photoelectric conversion element of the present invention.

FIG. 3 is a schematic sectional view showing an example of a photoelectric conversion element of the present invention.

FIG. 4 is a schematic sectional view showing an example of a photoelectric conversion element of the present invention.

FIG. 5 is a schematic sectional view showing an example of a laminated structure of a photoelectric conversion element in an image sensor of the present invention.

FIG. 6 is a schematic sectional view showing an example of a laminated structure of a photoelectric conversion element in an image sensor of the present invention.

### EMBODIMENT OF THE INVENTION

<Photoelectric Conversion Element>

A photoelectric conversion element of the present invention includes a first electrode, a second electrode, and at least one organic layer existing therebetween, the organic layer containing a first compound, and a second compound in which the maximum value of an absorption coefficient at a wavelength of 400 to 700 nm is 5×10⁴ cm⁻¹ or more, the first compound being represented by the following general formula (1).

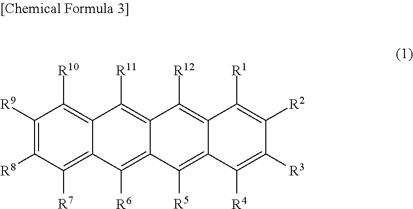

In the general formula (1), R¹ to R¹² may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, a carbonyl group, a carboxyl group, an oxycarbonyl group, a carbamoyl group, an amino group, a nitro group, a cyano group, a silyl group and —P(═O)R¹³R¹⁴. R¹³ and R¹⁴ each represent an aryl group or a heteroaryl group. Adjacent substituents may be linked together to form a ring structure.

R⁵ and R¹² in the general formula (1) each represent a group represented by the following general formula (2) or (3).

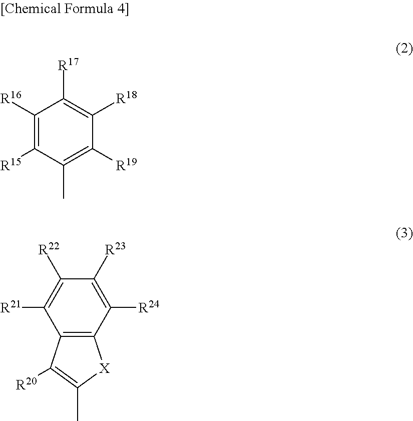

In the general formula (2) or (3), R¹⁵ to R²⁴ may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, a carbonyl group, a carboxyl group, an oxycarbonyl group, a carbamoyl group, an amino group, a nitro group, a cyano group, a silyl group and —P(═O)R¹³R¹⁴. R¹³ and R¹⁴ each represent an aryl group or a heteroaryl group. R¹⁶ to R¹⁹ _{and} R²¹ to R²⁴ may form a ring between adjacent substituents. X represents an oxygen atom, a sulfur atom or —NR²⁵. R²⁵ is hydrogen, an alkyl group, a cycloalkyl group, a heterocyclic group, an aryl group or a heteroaryl group.

Hereinafter, the “first compound represented by the general formula (1)” may be referred to as a “first compound”. In the present invention, the “second compound in which the maximum value of an absorption coefficient at a wavelength of 400 to 700 nm is 5×10⁴ cm⁻¹ or more” may be referred to as a “second compound” hereinafter.

Examples of the photoelectric conversion element of the present invention are shown in FIGS. 1 to 4.

FIG. 1 shows an example of a photoelectric conversion element including a first electrode 10, a second electrode 20, and one organic layer 11 interposed therebetween. The organic layer 11 in FIG. 1 is a photoelectric conversion element 15 which converts light into electric energy. The organic layer in the present invention is a layer containing an organic compound, and examples of the organic layer include photoelectric conversion layers and charge blocking layers.

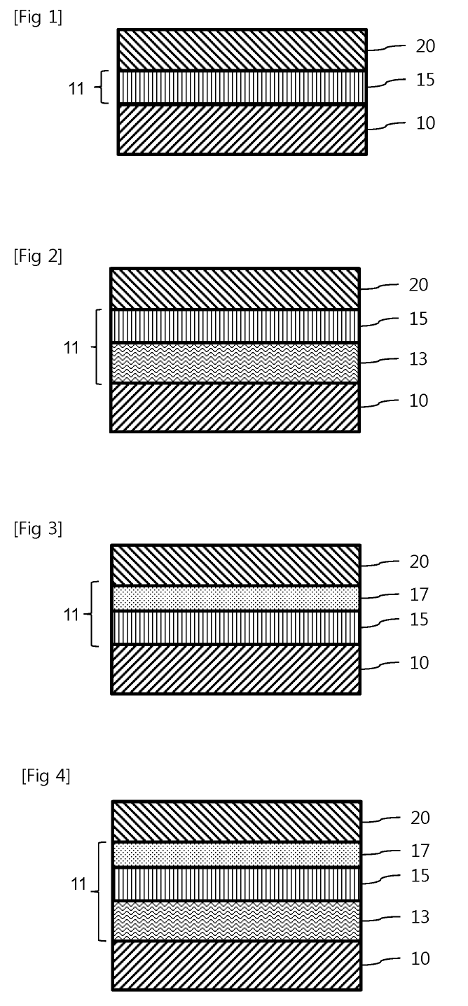

A description will be made below for FIGS. 2 to 4 by enumerating, as an example, the case where the first electrode 10 is an cathode and the second electrode 20 is an anode. As shown in FIGS. 2 to 4, a charge blocking layer may be inserted, in addition to a photoelectric conversion layer, between the cathode and the anode. The charge blocking layer is a layer that serves to block electrons or holes. The charge blocking layer serves as an electron blocking layer 13 when inserted between the cathode and the photoelectric conversion layer, and serves as a hole blocking layer 17 when inserted between the anode and the photoelectric conversion layer 15. The photoelectric conversion element may include only one of these charge blocking layers (FIGS. 2 and 3), or both of these charge blocking layers (FIG. 4).

Further, when the photoelectric conversion layer is composed of two or more photoelectric conversion materials, the photoelectric conversion layer may be a single layer in which two or more photoelectric conversion materials are mixed, or a plurality of layers in which layers composed of one or more photoelectric conversion material(s) are laminated. Furthermore, the photoelectric conversion layer may have a structure in which a mixed layer is mixed with each single layer.

(First Compound)

The first compound represented by the general formula (1) in the present invention will be described.

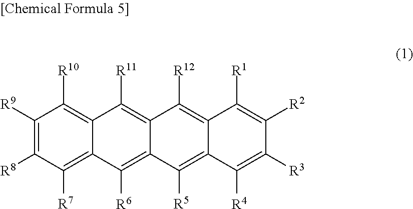

In the general formula (1), R¹ to R¹² may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, a carbonyl group, a carboxyl group, an oxycarbonyl group, a carbamoyl group, an amino group, a nitro group, a cyano group, a silyl group and —P(═O)R¹³R¹⁴. R¹³ and R¹⁴ each represent an aryl group or a heteroaryl group. Adjacent substituents may be linked together to form a ring structure.

In the present invention, hydrogen may include deuterium.

The alkyl group represents, for example, a saturated aliphatic hydrocarbon group such as a methyl group, an ethyl group, an n-propyl group, an isopropyl group, an n-butyl group, a sec-butyl group, or a tert-butyl group, and optionally has a substituent. There is no particular limitation on additional substituent when the alkyl group is substituted, and examples thereof include an alkyl group, an aryl group, a heteroaryl group, and the like. This aspect is common for the additional substituent when each substituent such as a cycloalkyl group or a heterocyclic group as described below is substituted. There is no particular limitation on the carbon number of the alkyl group, and the carbon number is usually in a range of 1 or more and 20 or less, and more preferably 1 or more and 8 or less, in view of availability and costs. When the alkyl group is substituted, the carbon number of the alkyl group includes the carbon number of the additional substituent. When each substituent such as a cycloalkyl group or a heterocyclic group as described below is substituted, the carbon number of each substituent includes the carbon number of the additional substituent.

The cycloalkyl group represents, for example, a saturated alicyclic hydrocarbon group such as cyclopropyl, cyclohexyl, norbornyl, or adamantyl, and optionally has a substituent. There is no particular limitation on the carbon number of the alkyl group moiety, and the carbon number is usually in a range of 3 or more and 20 or less.

The heterocyclic group represents, for example, an aliphatic ring having an atom other than carbon, such as a pyran ring, a piperidine ring or a cyclic amide, and optionally has a substituent. There is no particular limitation on the carbon number of the heterocyclic group, and the carbon number is usually in a range of 2 or more and 20 or less.

The alkenyl group represents, for example, an unsaturated aliphatic hydrocarbon group having a double bond, such as a vinyl group, an allyl group, or a butadienyl group, and optionally has a substituent. There is no particular limitation on the carbon number of the alkenyl group, and the carbon number is usually in a range of 2 or more and 20 or less.

The cycloalkenyl group represents, for example, an unsaturated aliphatic hydrocarbon group having a double bond, such as a cyclopentenyl group, a cyclopentadienyl group or a cyclohexenyl group, and optionally has a substituent. There is no particular limitation on the carbon number of the cycloalkenyl group, and the carbon number is usually in a range of 2 or more and 20 or less.

The alkynyl group represents, for example, an unsaturated aliphatic hydrocarbon group having a triple bond, such as an ethynyl group, and optionally has a substituent. There is no particular limitation on the carbon number of the alkynyl group, and the carbon number is usually in a range of 2 or more and 20 or less.

The alkoxy group represents, for example, a functional group in which aliphatic hydrocarbon groups are bonded through an ether bond, such as a methoxy group, an ethoxy group, or a propoxy group, and this aliphatic hydrocarbon group optionally has a substituent. There is no particular limitation on the carbon number of the alkoxy group, and the carbon number is usually in a range of 1 or more and 20 or less.

The alkylthio group is a group in which the oxygen atom of the ether bond in the alkoxy group is replaced by a sulfur atom. The hydrocarbon group in the alkylthio group optionally has a substituent. There is no particular limitation on the carbon number of the alkylthio group, and the carbon number is usually in a range of 1 or more and 20 or less.

The aryl ether group represents, for example, a functional group in which aromatic hydrocarbon groups are bonded through an ether bond, such as a phenoxy group, and the aromatic hydrocarbon group optionally has a substituent. There is no particular limitation on the carbon number of the aryl ether group, and the carbon number is usually in a range of 6 or more and 40 or less.

The aryl thioether group is a group in which the oxygen atom of the ether bond in the aryl ether group is replaced by a sulfur atom. The aromatic hydrocarbon group in the aryl ether group optionally has a substituent. There is no particular limitation on the carbon number of the aryl ether group, and the carbon number is usually in a range of 6 or more and 40 or less.

The aryl group represents, for example, an aromatic hydrocarbon group such as a phenyl group, a naphthyl group, a biphenyl group, a fluorenyl group, a phenanthryl group, a triphenylenyl group, or a terphenyl group. The aryl group optionally has a substituent. There is no particular limitation on the carbon number of the aryl group, and the carbon number is usually in a range of 6 or more and 40 or less.

The heteroaryl group represents a cyclic aromatic group having one or plural atom (s) other than carbon in the ring, such as a furanyl group, a thiophenyl group, a pyridyl group, a quinolinyl group, a pyrazinyl group, a pyrimidinyl group, a triazinyl group, a naphthylidyl group, a benzofuranyl group, a benzothiophenyl group, or an indolyl group, and the heteroaryl group optionally has a substituent. There is no particular limitation on the carbon number of the heteroaryl group, and the carbon number is usually in a range of 2 or more and 30 or less.

The halogen represents fluorine, chlorine, bromine, or iodine.

The amino group optionally has a substituent. Examples of the substituent include an aryl group and a heteroaryl group, and these substituents may be further substituted.

The silyl group represents, for example, a functional group having a bond to a silicon atom, such as a trimethylsilyl group, and optionally has a substituent. There is no particular limitation on the carbon number of the silyl group, and the carbon number is usually in a range of 3 or more and 20 or less. The silicon number is usually in a range of 1 or more and 6 or less.

—P(═O)R¹¹R¹² optionally has a substituent. Examples of the substituent include an aryl group and a heteroaryl group, and these substituents may be further substituted.

Any two adjacent substituents (e.g. R¹ and R² in the general formula (1)) may be linked together to form a conjugated or unconjugated fused ring. Particularly, formation of a structure in which five fused rings are formed as a whole with R¹ and R² forming a ring is preferable because charge mobility is improved. As the structure in which five fused rings are formed as a whole, benzo [a] naphthacene is especially preferable. As constituent elements of the fused ring, an element selected from nitrogen, oxygen, sulfur, phosphorus and silicon may exist in addition to carbon. The fused ring may be further fused with another ring.

R⁵ and R¹² in the general formula (1) each represent a group represented by the general formula (2) or (3).

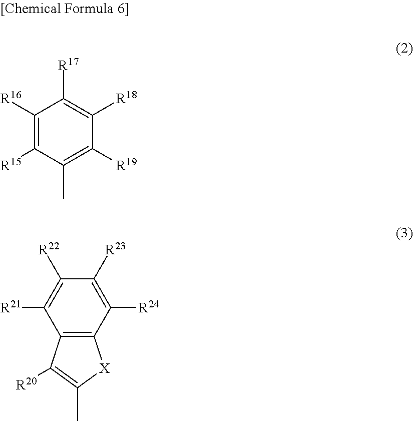

In the general formula (2) or (3), R¹⁵ to R²⁴ may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, a carbonyl group, a carboxyl group, an oxycarbonyl group, a carbamoyl group, an amino group, a nitro group, a cyano group, a silyl group and —P(═O)R¹³R¹⁴. R¹³ and R¹⁴ each represent an aryl group or a heteroaryl group. R¹⁶ to R¹⁹ and R²¹ to R²⁴ may form a ring between adjacent substituents. X represents an oxygen atom, a sulfur atom or —NR²⁵. R²⁵ is hydrogen, an alkyl group, a cycloalkyl group, a heterocyclic group, an aryl group or a heteroaryl group.

It is preferable that as described above, two groups represented by the general formula (2) or (3) exist at specific bonding positions (5-position and 12-position) in a naphthacene skeleton because both high charge mobility and high heat resistance can be achieved, so that the photoelectric conversion efficiency and durability of the photoelectric conversion element can be improved.

A compound having a group represented by the general formula (2) has high charge mobility because the compound has an aryl group, so that charge transfer between molecules with π electrons is smoothed. Therefore, the compound considerably contributes to an improvement in external quantum efficiency. It is preferable that among groups represented by the general formula (2), R¹⁵ is an alkyl group, an alkoxy group, an aryl group or a heteroaryl group because the molecular interaction between naphthacene skeletons is suppressed, so that high photoelectric conversion efficiency can be achieved, and a stable thin film can be formed. In particular, it is more preferable that R¹⁵ is an alkyl group or alkoxy group with a carbon number of 1 to 20, or an aryl group or heteroaryl group with a carbon number of 4 to 14 because acquirement of raw materials and the synthesis process become easy, so that costs can be reduced. Further, it is especially preferable that a naphthalene ring is formed as a whole with R¹⁷ and R¹⁸ forming a ring because extremely excellent charge mobility is achieved to contribute to an improvement in external quantum efficiency.

A compound having a group represented by the general formula (3) is preferable in that heat resistance is improved because the compound has a bicyclic benzoheterocyclic ring, so that a high glass transition temperature (Tg) can be secured. It is preferable that among groups represented by the general formula (3), R²° is an alkyl group, an alkoxy group, an aryl group or a heteroaryl group because the molecular interaction between naphthacene skeletons is suppressed, so that high photoelectric conversion efficiency can be achieved, and a stable thin film can be formed. In particular, it is more preferable that R²° is an alkyl group or alkoxy group with a carbon number of 1 to 20, or an aryl group or heteroaryl group with a carbon number of 4 to 14 because acquirement of raw materials and the synthesis process become easy, so that costs can be reduced.

Examples of the alkyl group or alkoxy group with a carbon number of 1 to 20 include a methyl group, an ethyl group, a n-propyl group, an isopropyl group, a n-butyl group, a sec-butyl group, a tert-butyl group, a n-pentyl group, a cyclopentyl group, a n-hexyl group, a cyclohexyl group, an adamantyl group, a methoxy group, an ethoxy group, a n-propyloxy group, an isopropyloxy group, a n-butoxy group, a sec-butoxy group, a tert-butoxy group, a n-pentoxy group, a cyclopentoxy group, a n-hexyloxy group and a cyclohexyloxy group. Among them, a methyl group, an ethyl group, a n-propyl group, an isopropyl group, a n-butyl group, a tert-butyl group and a methoxy group are preferable for securing both high photoelectric conversion efficiency and thin film stability and ease of acquirement of raw materials and the synthesis process.

Examples of the aryl group or heteroaryl group with a carbon number of 4 to 14 include a phenyl group, a naphthyl group, a phenanthryl group, an anthracenyl group, a fluorenyl group, a furanyl group, a thiophenyl group, a pyrrolyl group, a benzofuranyl group, a benzothiophenyl group, an indolyl group, a benzoxazolyl group, a benzothiazolyl group, a benzimidazolyl group, a pyridyl group, a quinolinyl group, a quinoxalinyl group, a carbazolyl group and a venatololyl group. Among them, a phenyl group, a naphthyl group, a phenanthryl group, a fluorenyl group, a benzofuranyl group, a benzothiophenyl group, a pyridyl group, a quinolinyl group and a quinoxalinyl group for securing both high photoelectric conversion efficiency and thin film stability and ease of acquirement of raw materials and the synthesis process.

The aryl group and heteroaryl group may further have a substituent. As examples of the substituent here, alkyl groups such as a methyl group, an ethyl group, a propyl group and a tert-butyl group, alkoxy groups such as a methoxy group and an ethoxy group, aryl ether groups such as a phenoxy group, aryl groups such as a phenyl group, a naphthyl group and a biphenyl group, and heteroaryl groups such as a pyridyl group, a quinolinyl group, a benzofuranyl group and a benzothiophenyl group are preferable. Among them, a methyl group, a tert-butyl group and a phenyl group are especially preferable from the viewpoint of ease of acquirement of raw materials and the synthesis process.

X in the general formula (3) is preferably an oxygen atom because higher photoelectric conversion efficiency is obtained.

R¹ to R⁴, R⁶ to R¹¹, R¹⁶ to R¹⁹ and R²¹ to R²⁴ are each preferably hydrogen or deuterium because vapor deposition becomes easier as the molecular weight of the first compound decreases.

A known method can be used for synthesis of the first compound represented by the general formula (1). Examples of the method for introducing a group represented by the general formula (2) or (3) into the naphthacene skeleton in the first compound include, but are not limited to, a method using a coupling reaction of a naphthoquinone derivative with an organic metal reagent, and a method using a coupling reaction of a halogenated naphthacene derivative with a boronic acid reagent under a palladium or nickel catalyst.

Specific examples of the first compound represented by the above general formula (1) may include the following compounds.

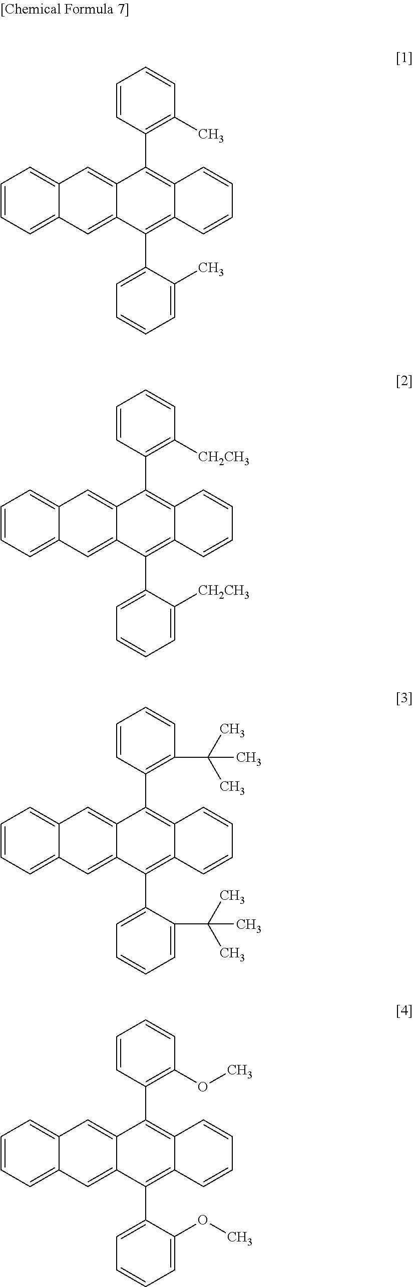

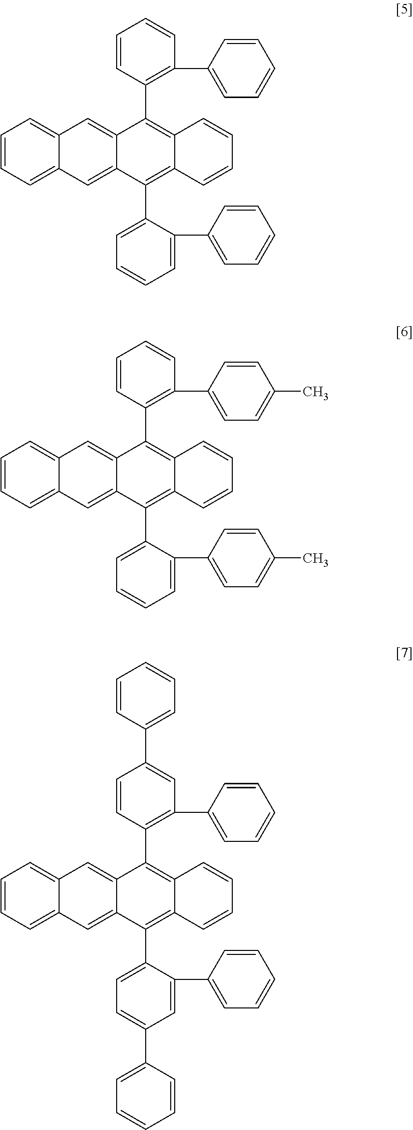

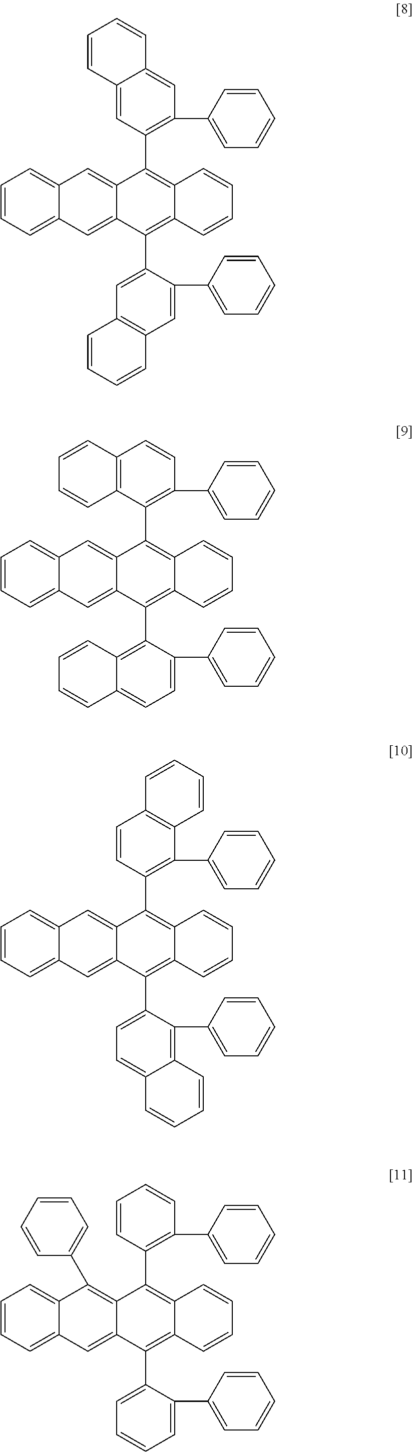

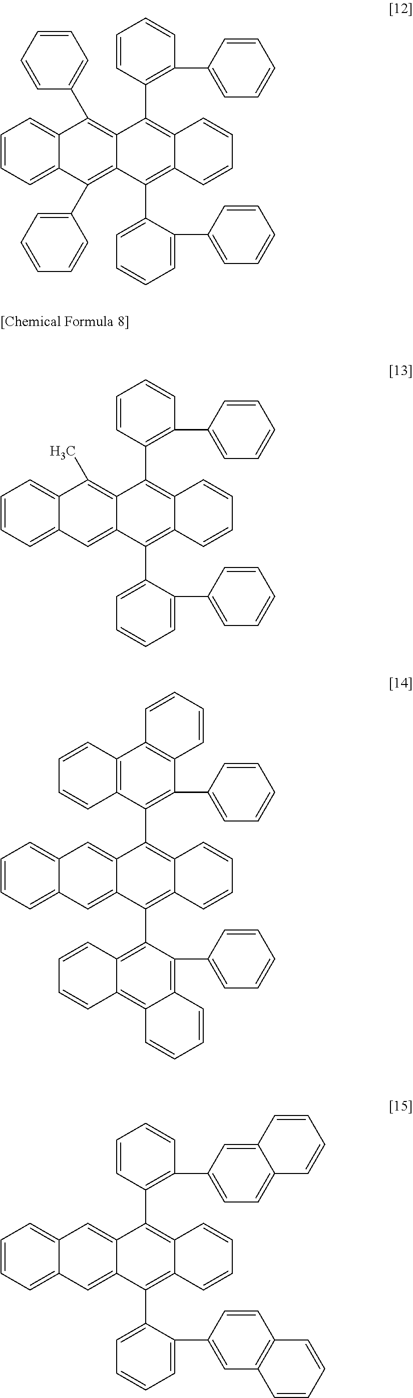

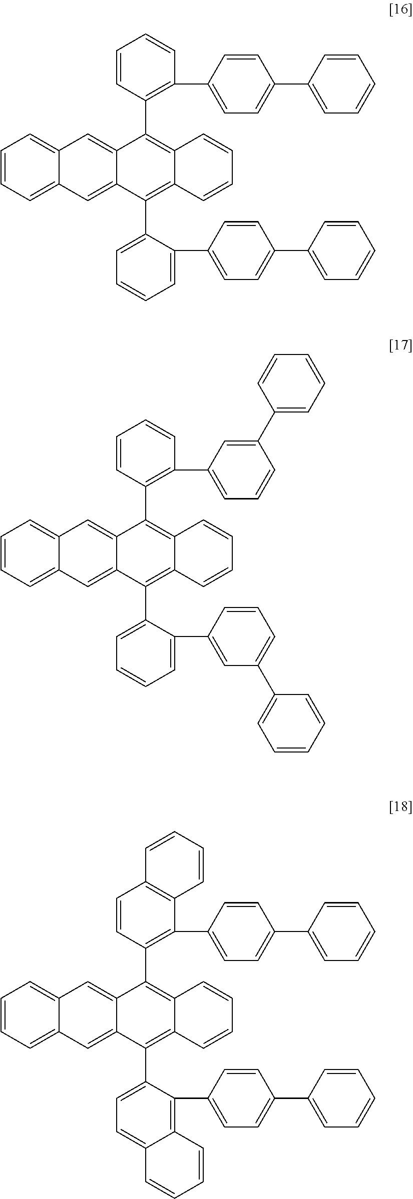

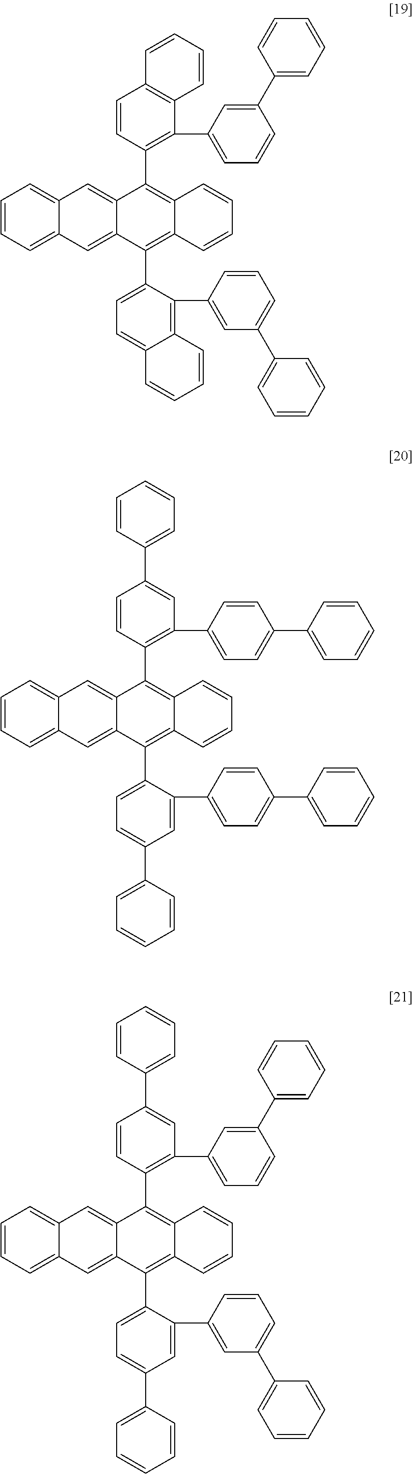

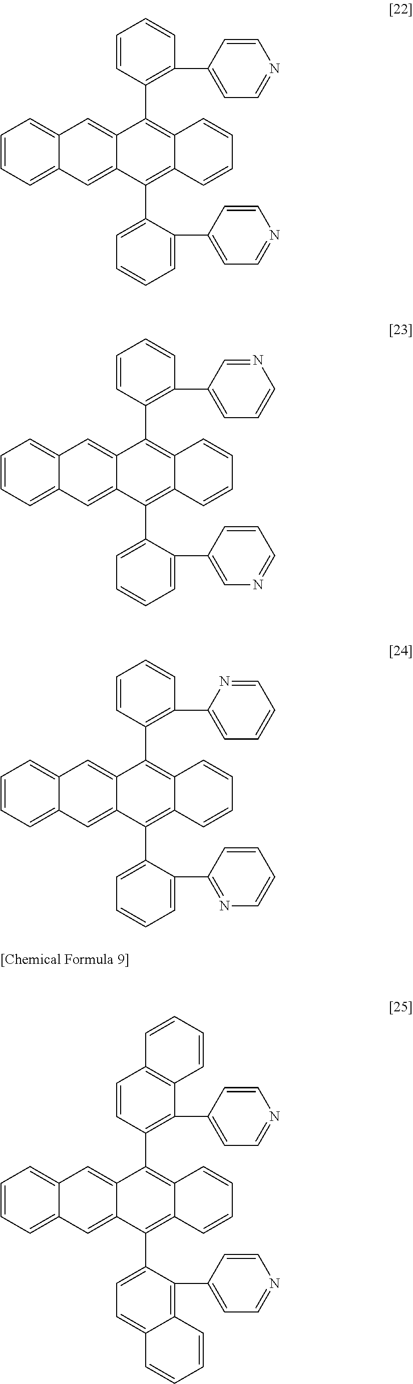

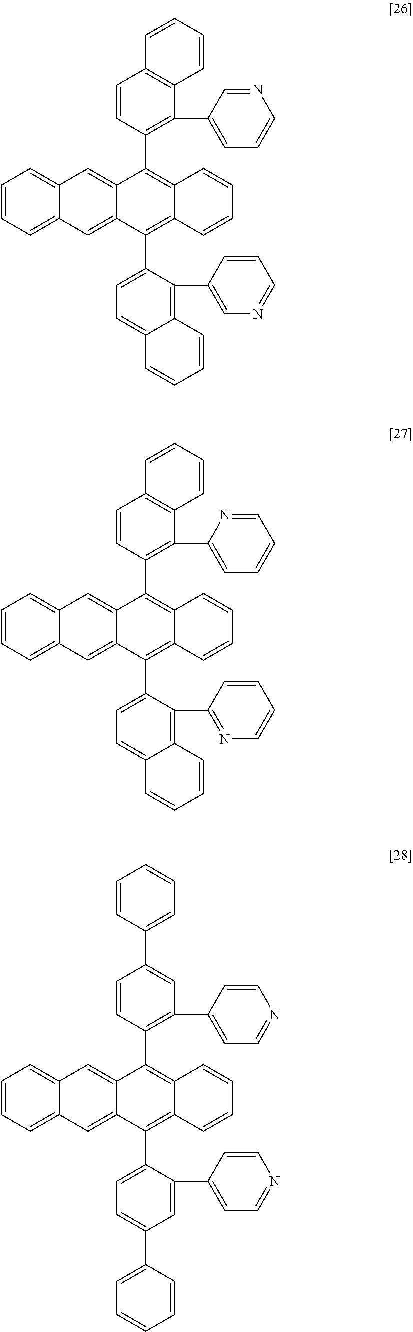

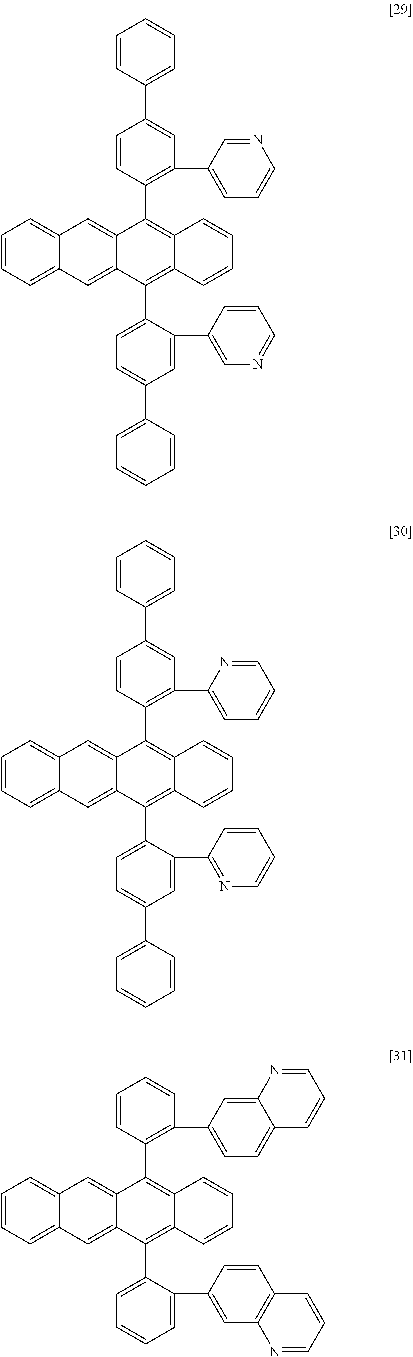

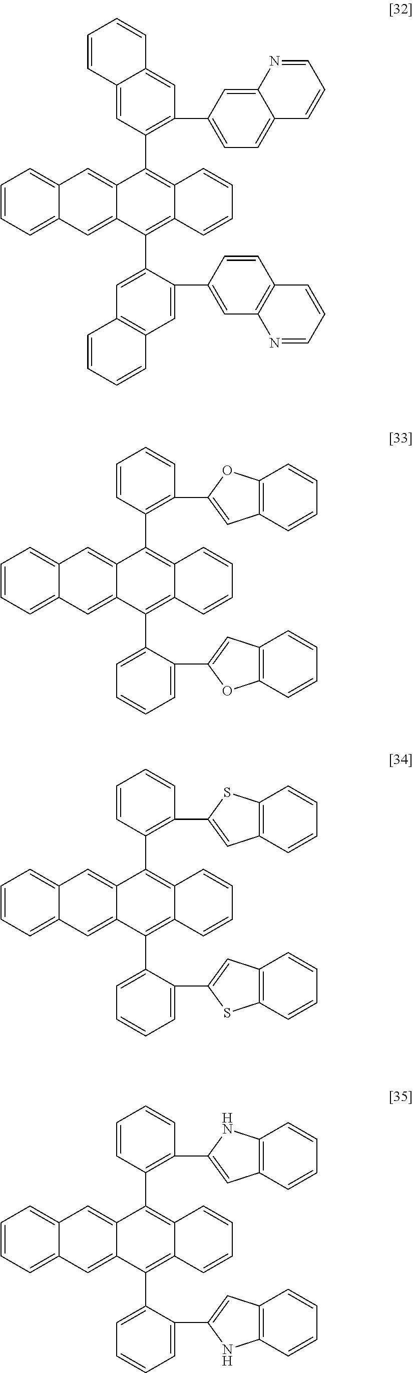

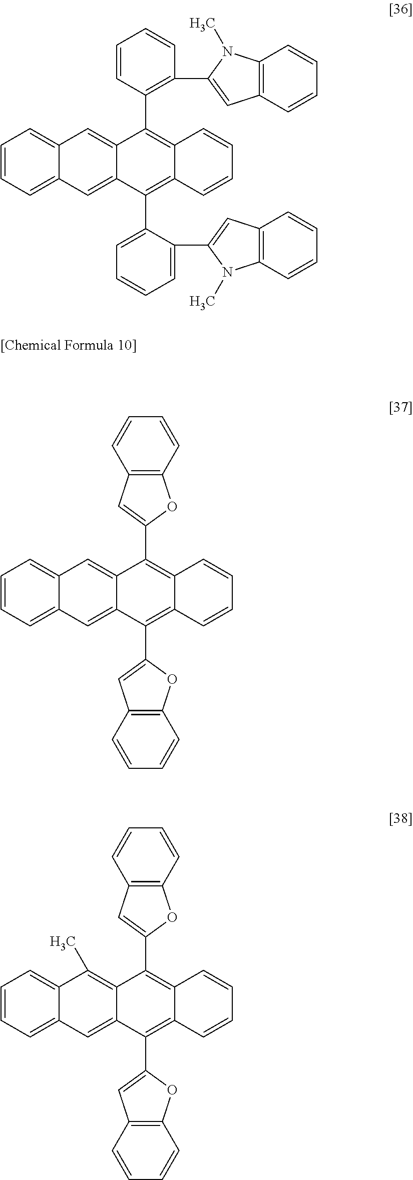

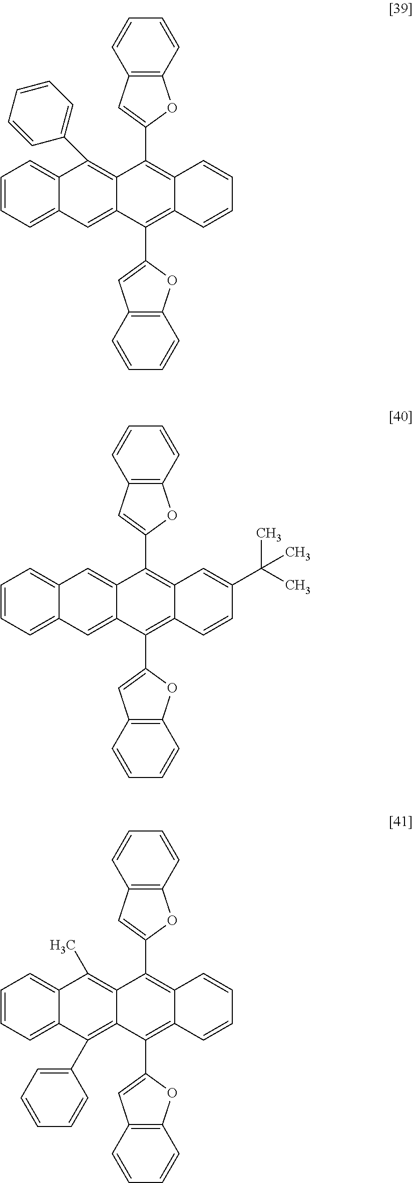

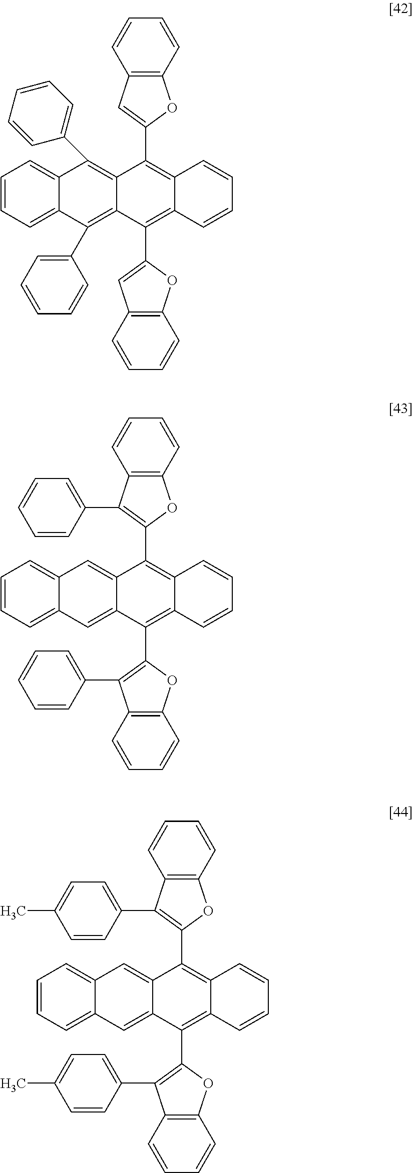

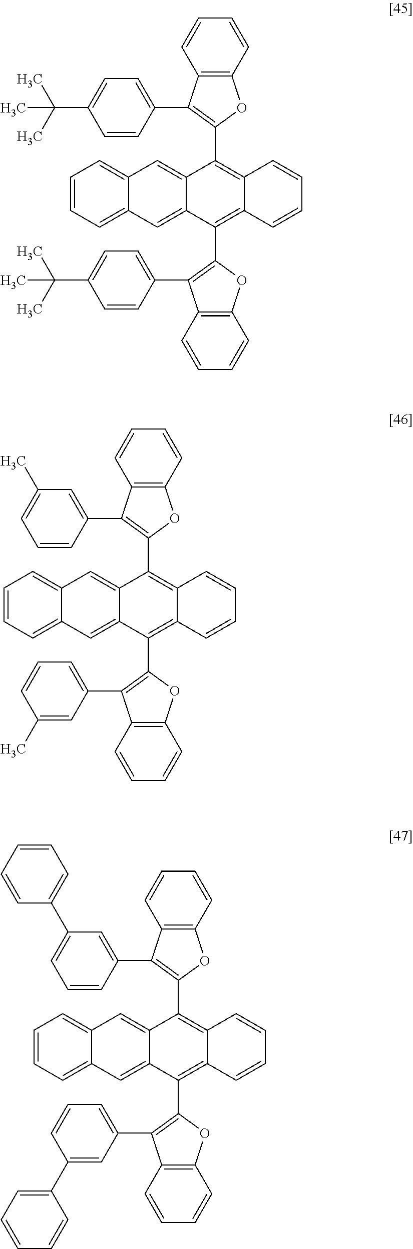

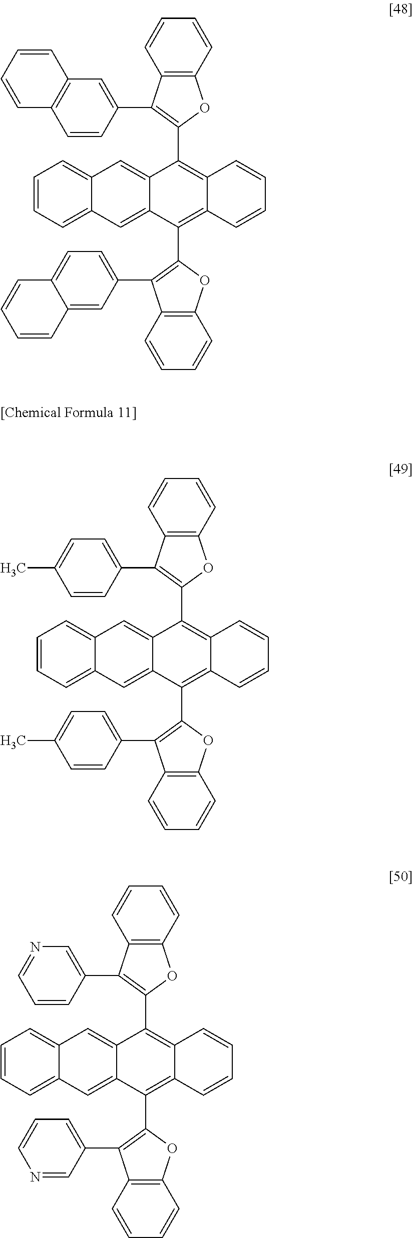

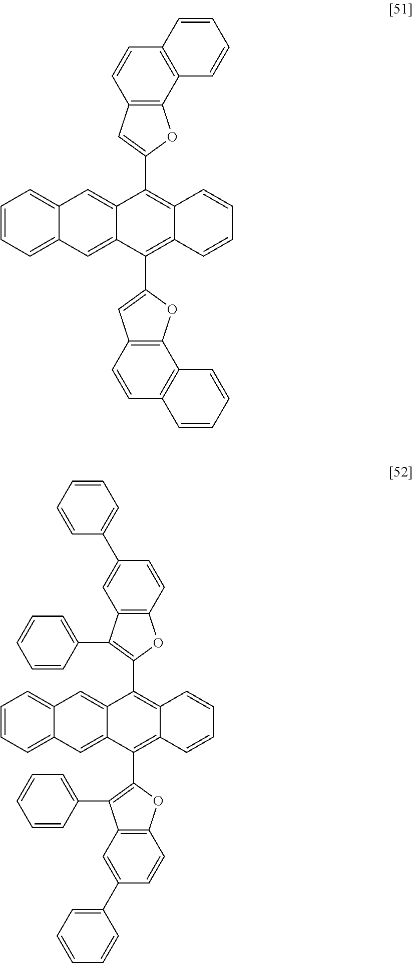

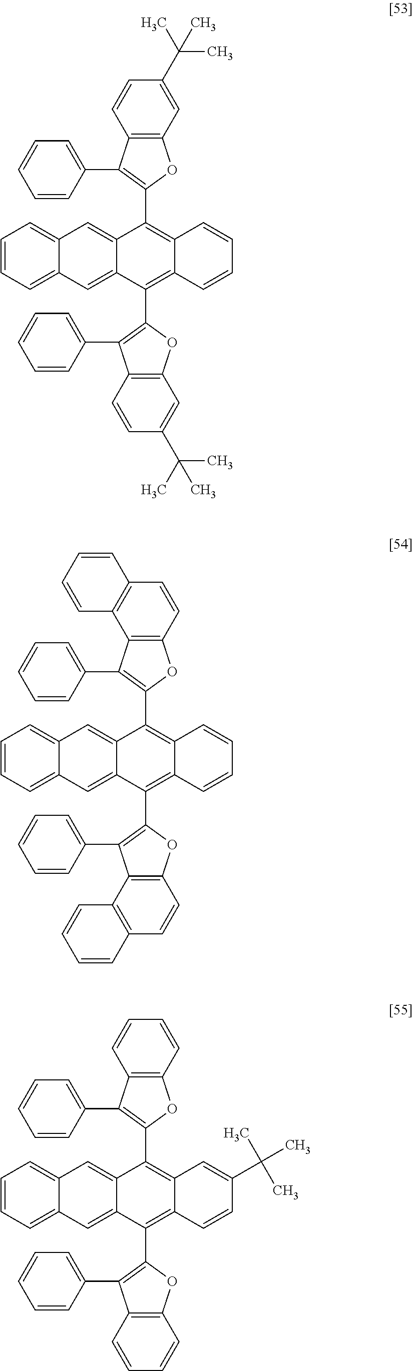

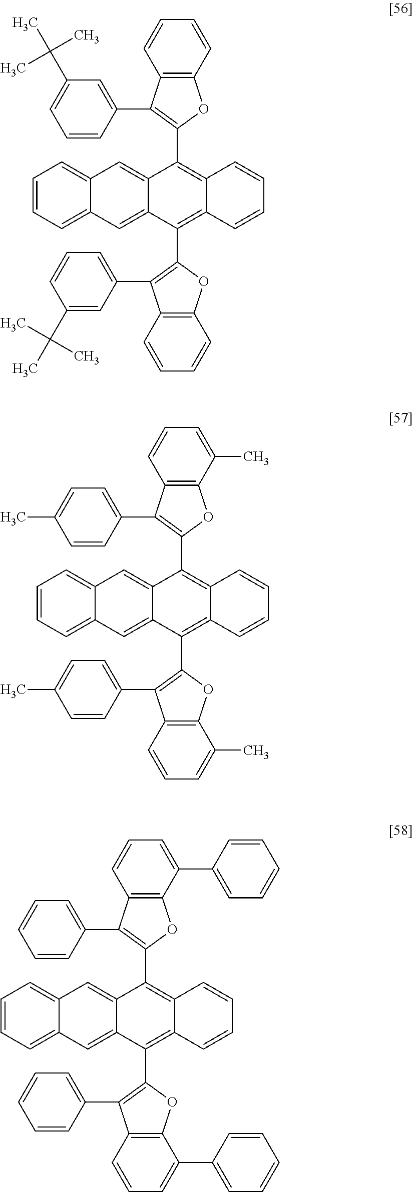

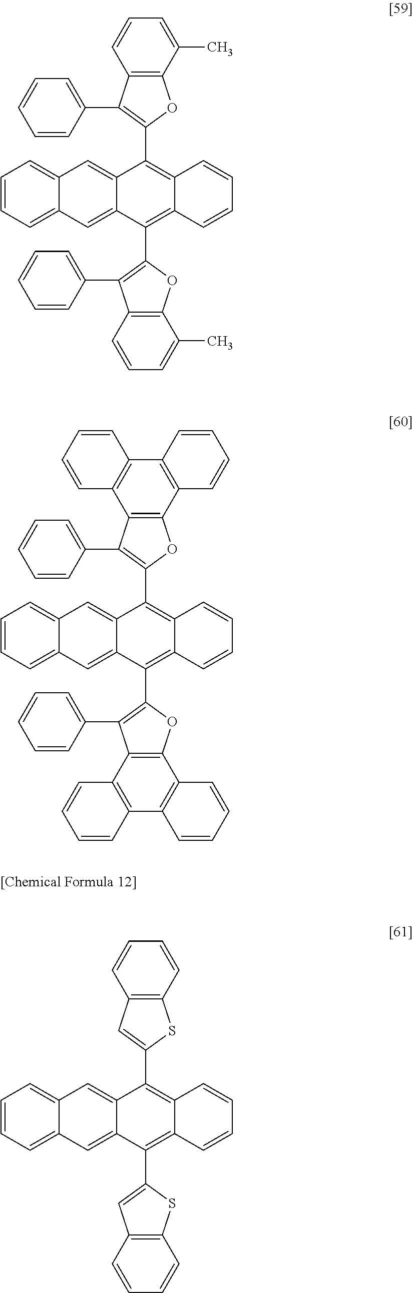

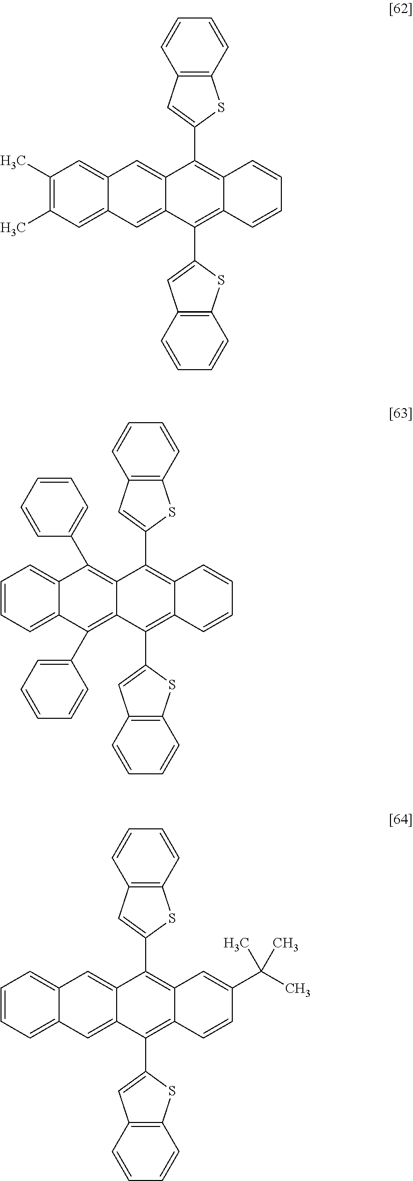

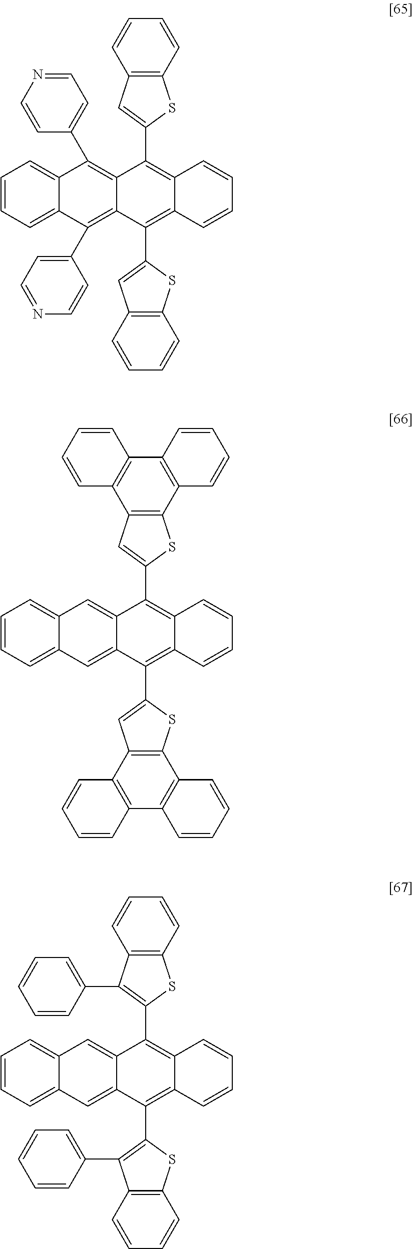

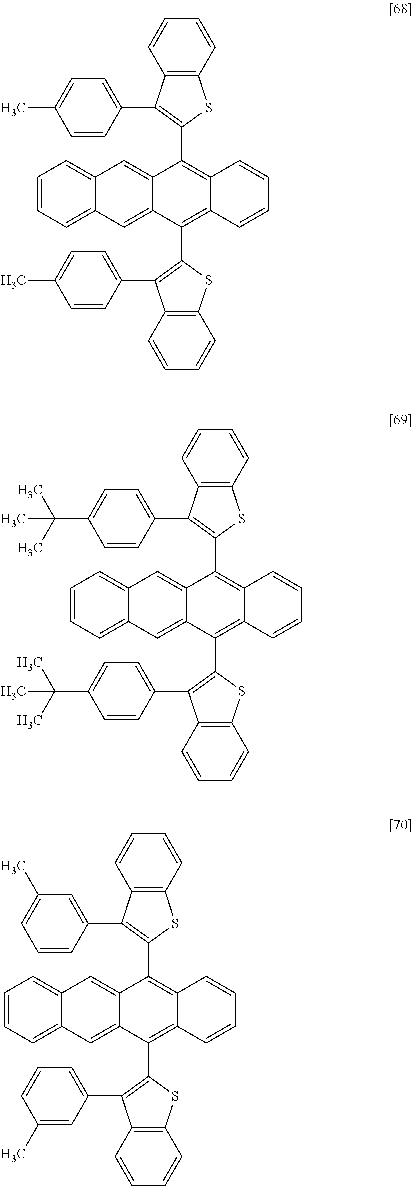

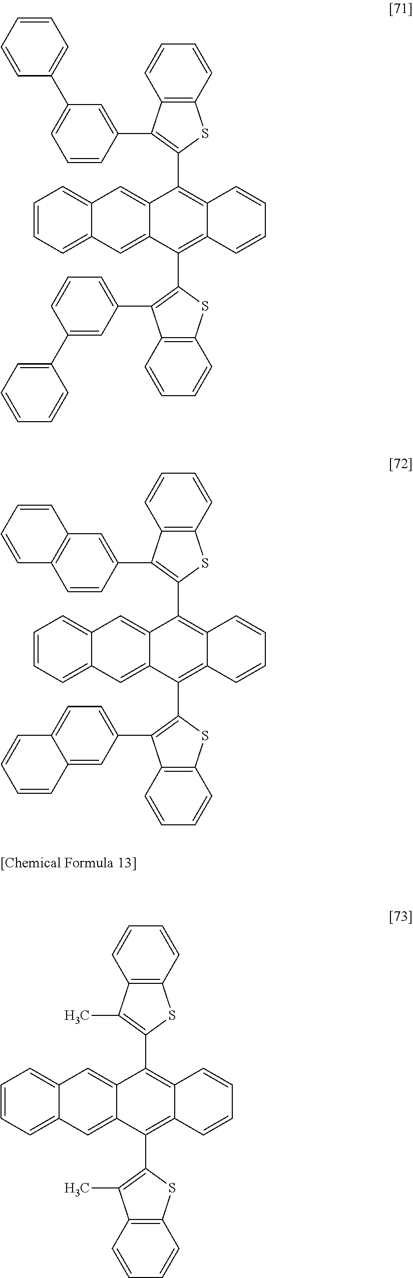

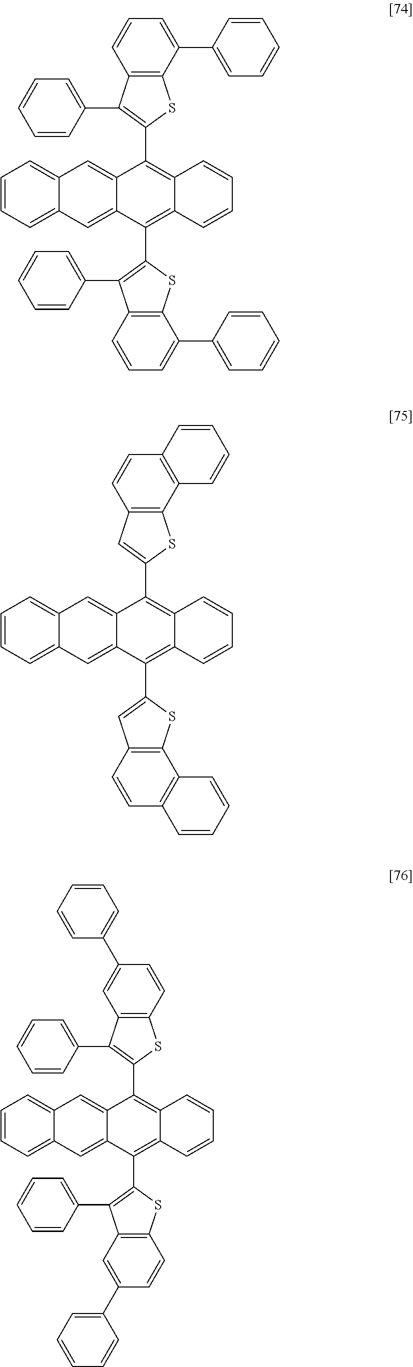

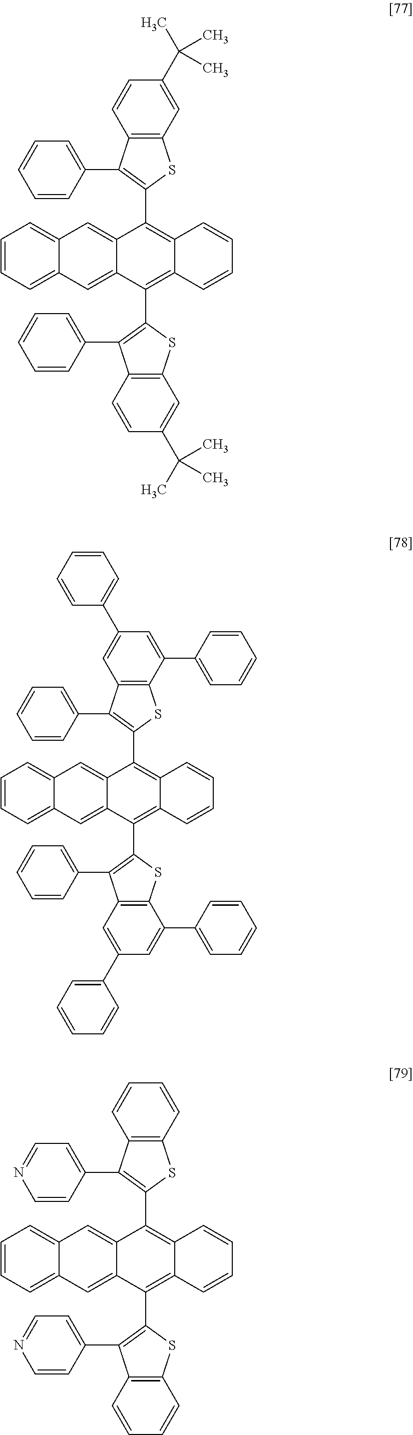

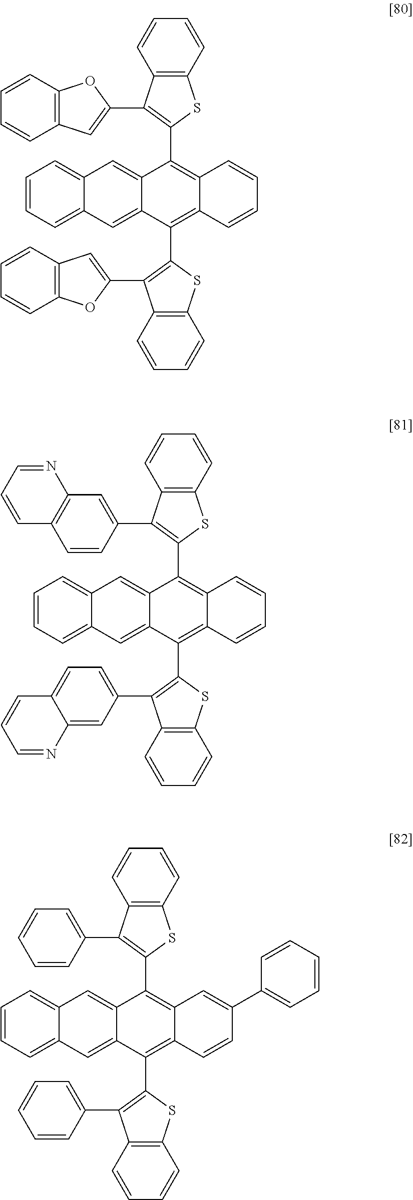

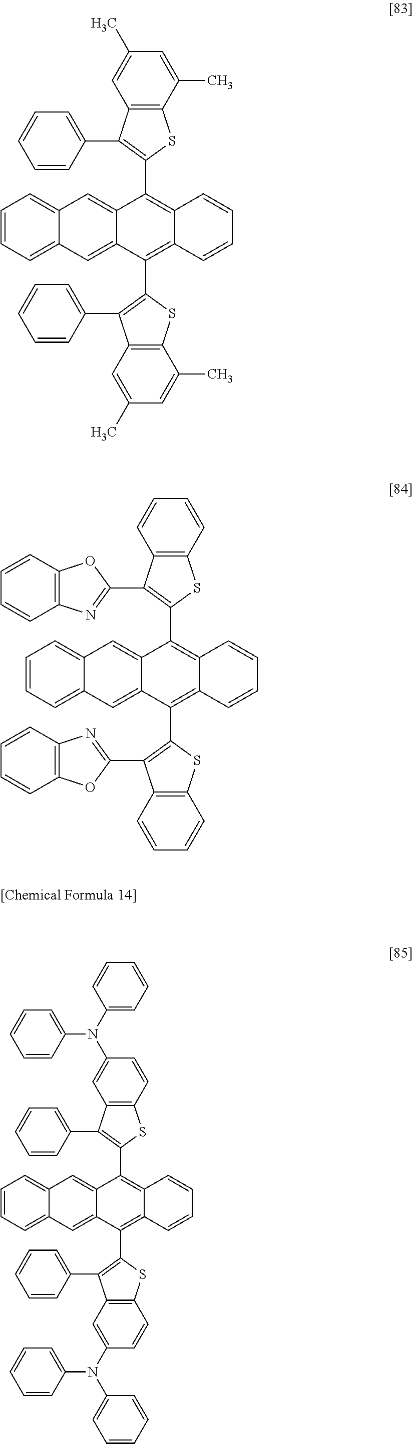

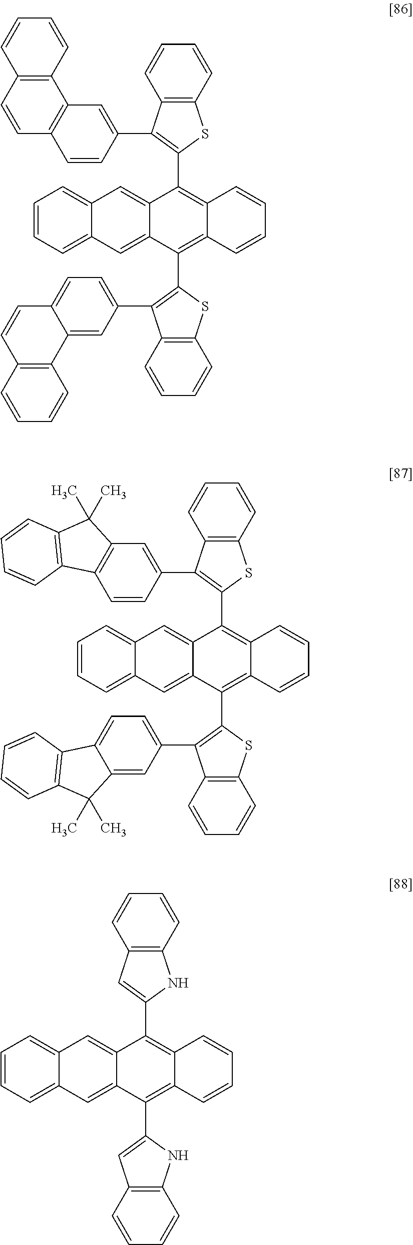

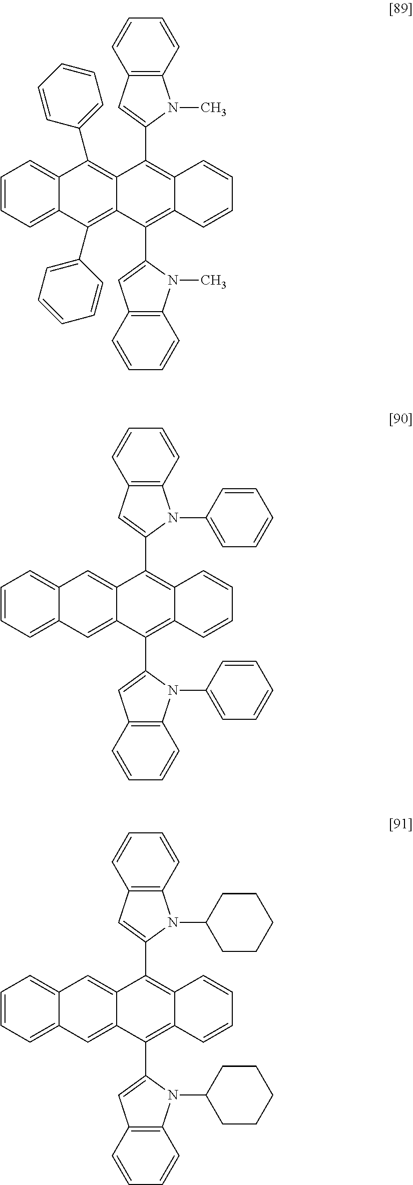

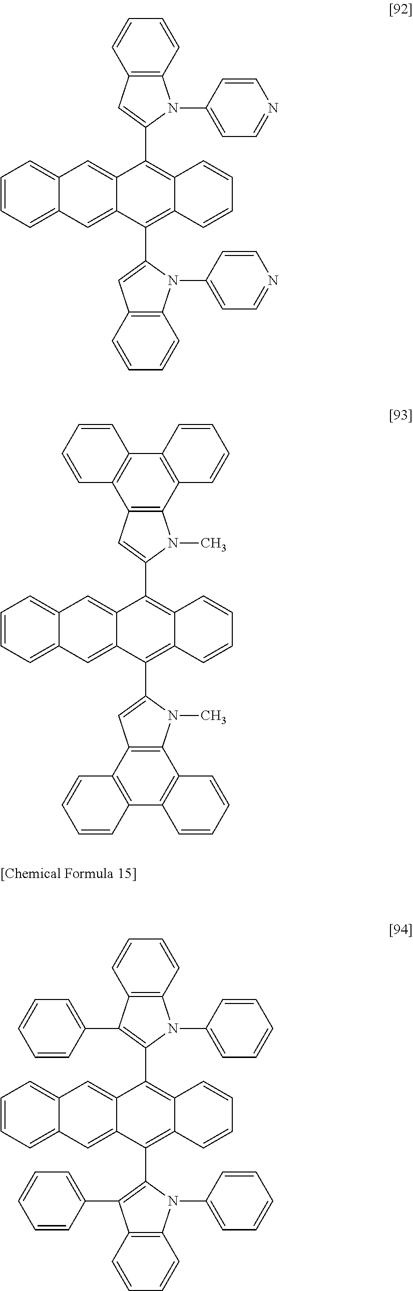

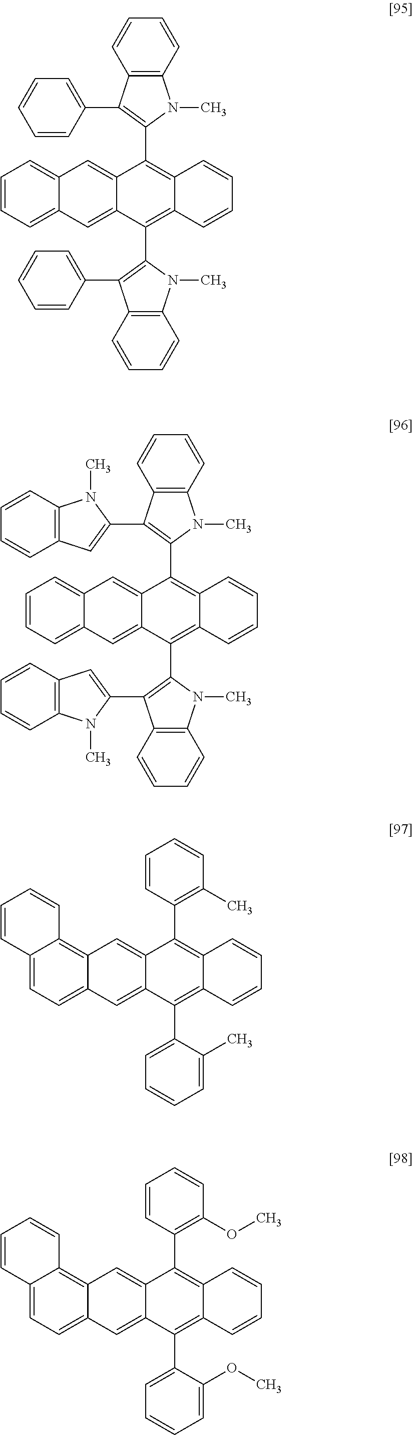

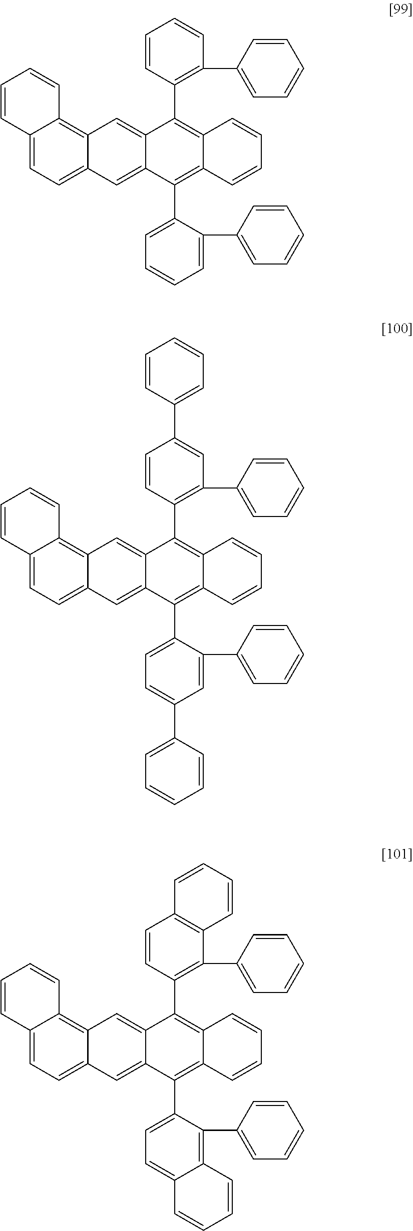

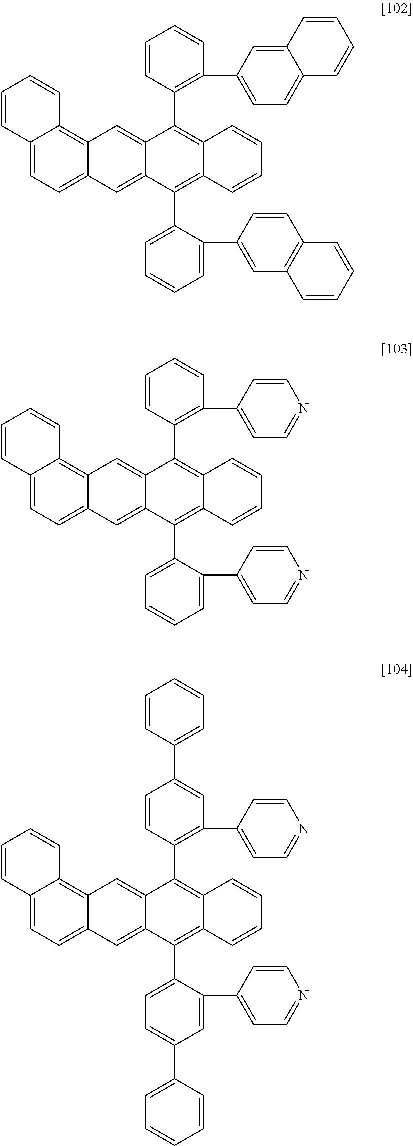

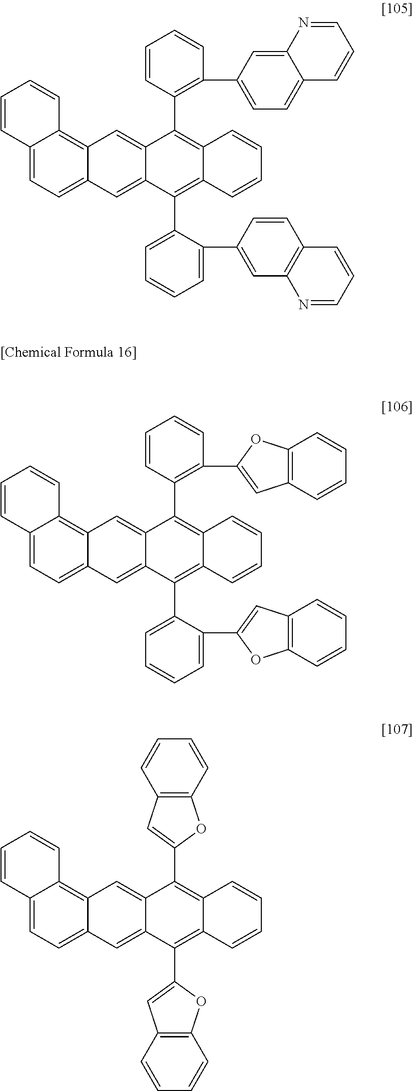

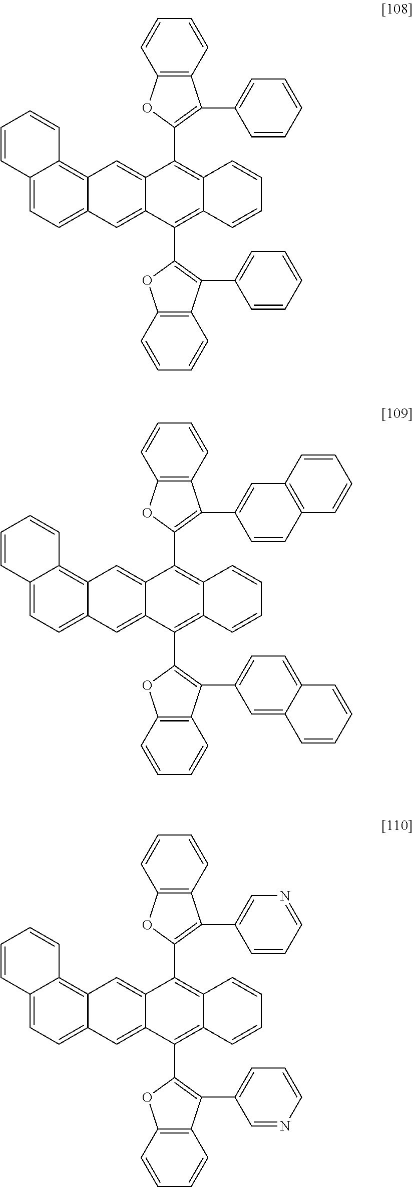

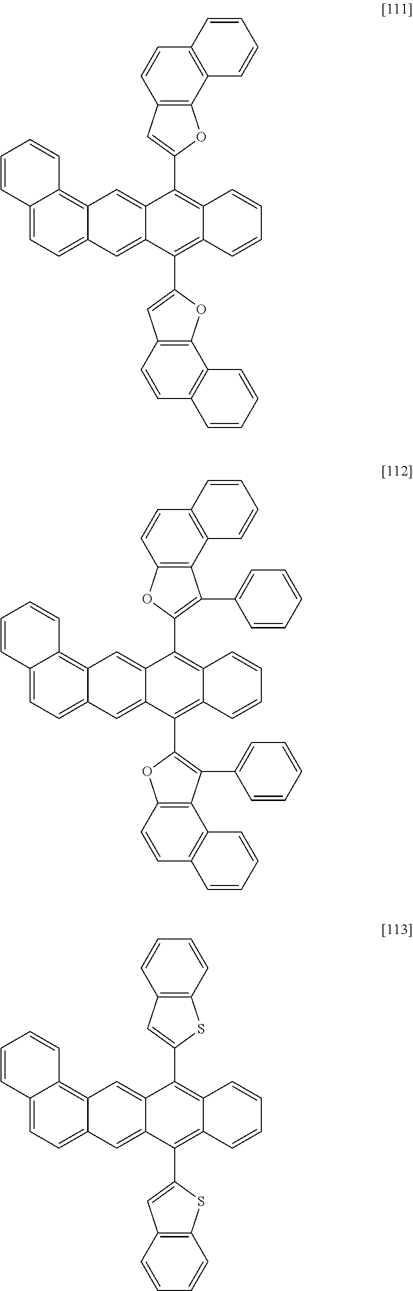

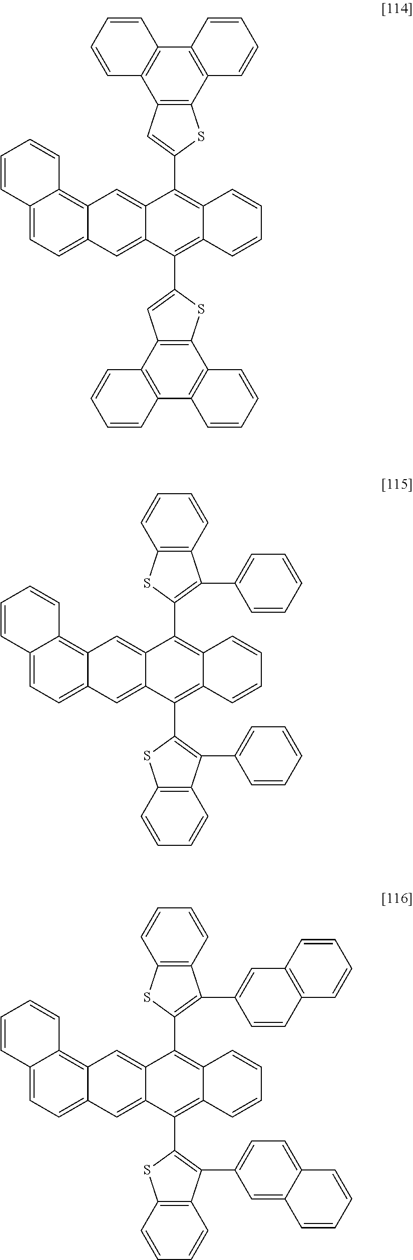

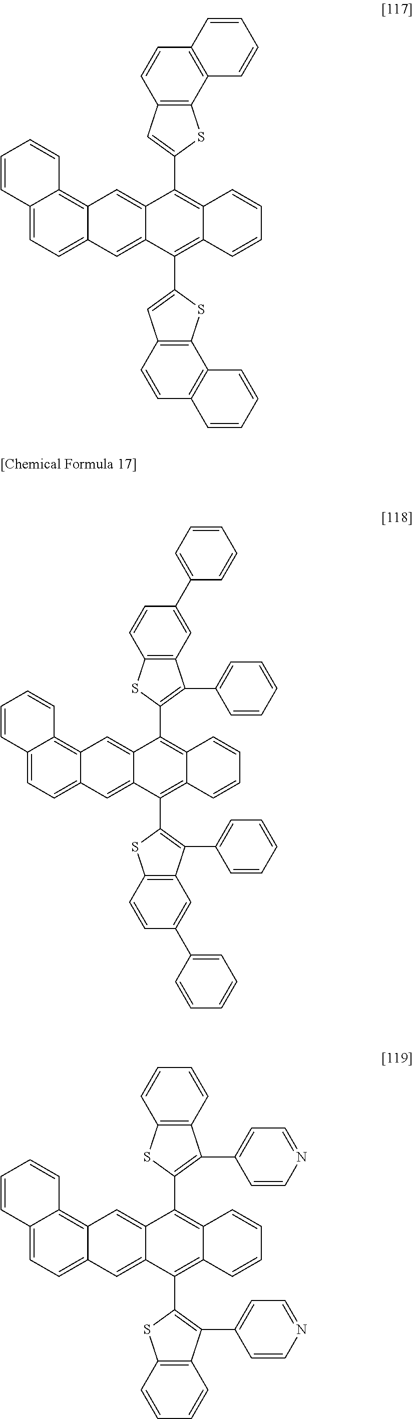

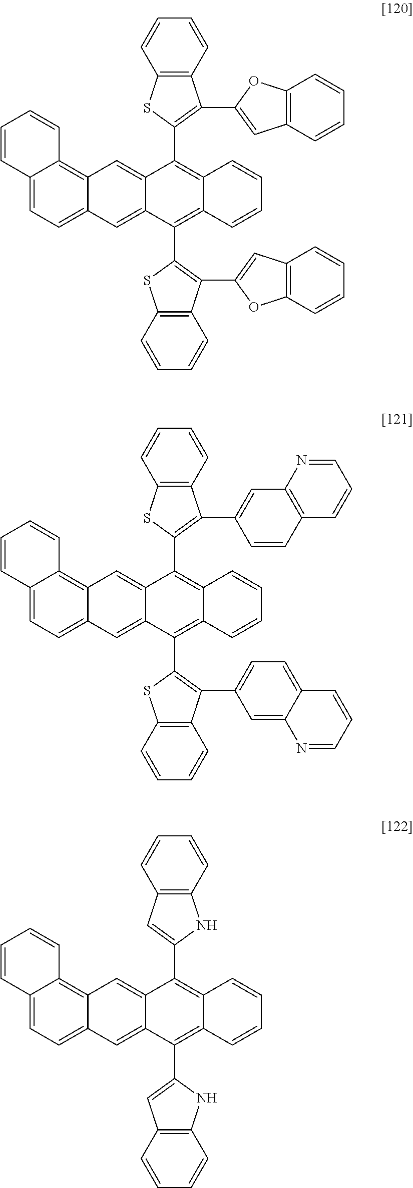

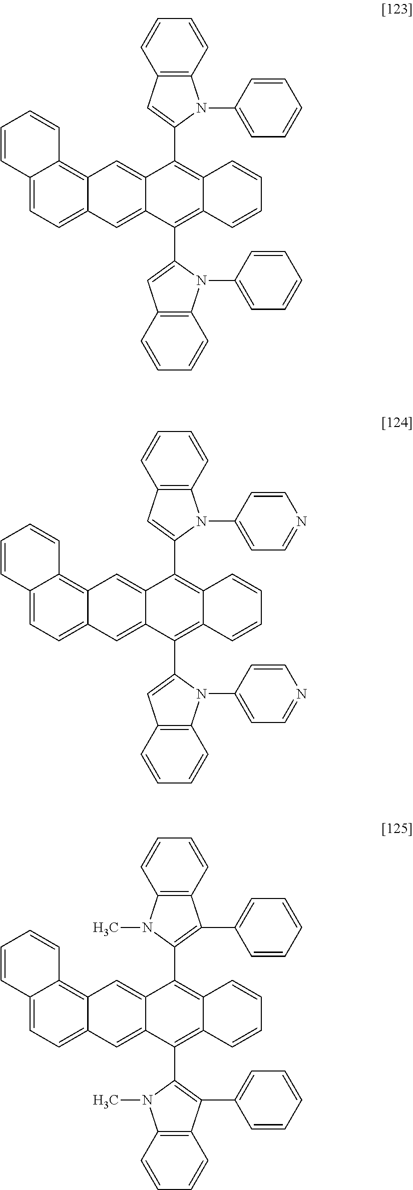

(Second Compound)

The second compound in which the maximum value of an absorption coefficient at a wavelength of 400 to 700 nm is 5×10⁴ cm⁻¹ or more in the present invention will be described. When two or more maximum values of an absorption coefficient exist at a wavelength of 400 to 700 nm, the maximum value of an absorption coefficient, which is the largest of these maximum values, is employed to make a judgement.

The first compound represented by the general formula (1) has high charge mobility, and is therefore excellent in capability of efficiently transporting generated charges to an electrode, but on the other hand, the first compound has a small absorption coefficient. Specifically, the absorption coefficient of the first compound represented by the general formula (1), depending on a type of substituent to be introduced into a naphthacene skeleton, is 1×10⁴cm⁻¹ to 5×10⁴ cm⁻¹. This is almost the same value as the absorption coefficient (about 10⁴ cm⁻¹) of an inorganic thin film of silicon crystals etc. Therefore, the first compound represented by the general formula (1) cannot singly absorb incident light sufficiently, and thus most of the light is transmitted, so that an optical loss occurs, resulting in reduction of photoelectric conversion efficiency.

Meanwhile, as organic compounds to be used in the photoelectric conversion layer, many compounds having a large absorption coefficient of about 10⁵ to 10⁶ cm⁻¹ are known. For example, the compound A-1 shown below as an example has an absorption coefficient of 1.16×10⁵ cm⁻¹.

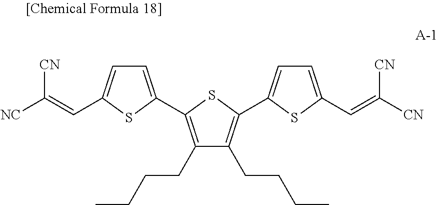 [SMILES: CCCCc1c(-c2ccc(C=C(C#N)C#N)s2)sc(-c2ccc(C=C(C#N)C#N)s2)c1CCCC]

Thus, high photoelectric conversion performance can be achieved by employing a structure in which the organic layer includes the first compound represented by the general formula (1), and the second compound in which the maximum value of an absorption coefficient at a wavelength of 400 to 700 nm is 5×10⁴ cm⁻¹ or more. Namely, when the second compound having a large absorption coefficient has a role of light absorption, and both the first compound and the second compound have a role of charge transfer, both light absorption property and charge mobility can be secured, and therefore photoelectric conversion performance can be exhibited.

Preferably, these compounds are contained particularly in the photoelectric conversion layer in the organic layer. These compounds are not necessarily contained only in the photoelectric conversion layer. For example, the electron blocking layer and the hole blocking layer may contain the first compound and the second compound for improving the charge mobility of the layers or increasing the number of carriers generated in the layers, or the electron blocking layer and the hole blocking layer may contain the second compound for improving the light absorption property of the whole photoelectric conversion element.

The absorption coefficient of the second compound is preferably as large as possible. For obtaining efficiency for light utilization, which is higher than that of an inorganic photoelectric conversion element, by making use of high light absorption property that is specific to an organic photoelectric conversion element, the absorption coefficient is preferably 5×10⁴ cm⁻¹ or more, more preferably 8×10⁴ cm⁻¹ or more, still more preferably 1×10⁵ cm⁻¹ or more.

As a material having an absorption coefficient as described above, a pigment-based material is suitable because of good light absorption property. Specific examples thereof include derivatives of merocyanine, coumarin, nile red, rhodamine, oxazine, acridine, squarylium, diketo-pyrrolo-pyrrole, pyrromethene, pyrene, perylene, thiophene, phthalocyanine and so on. Further, when the photoelectric conversion element of the present invention is used as an image sensor, a material having a single absorption peak at a wavelength of 400 to 700 nm is suitably used. Specific examples of the material having an absorption as described above and having a large absorption coefficient of 1×10⁵ cm⁻¹ or more include thiophene derivatives, pyrene derivatives and perylene derivatives.

The thiophene derivative is preferably a compound represented by the general formula (4).

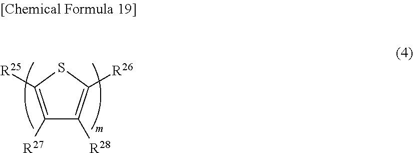

In the general formula (4), R²⁵ to R²⁸ may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, an amino group, a silyl group, —P(═O) R²⁹R³⁰, and a group represented by the following general formula (5). R²⁹ and R³⁰ each represent an aryl group or a heteroaryl group. m represents an integer of 1 to 6. At least one of R²⁵ to R²⁸ represents a group represented by the following general formula (5).

[Chemical Formula 20]

-LCN)ₙ (5)

In the general formula (5), n represents 1 or 2. L represents an alkenediyl group, an arylenediyl group or a heteroarylenediyl group when n represents 1. L represents an aklenetriyl group, an arylenetriyl group or a heteroarylenetriyl group when n represents 2.

The compound represented by the general formula (4) is a compound having a high light absorption coefficient, a single peak absorption, and good color selectivity. When m is an integer of 1 to 6, the compound has an absorption region in a wavelength range of 400 to 700 nm. For example, when a photoelectric conversion element having an absorption in a green region is produced, m is preferably 2 to 4, especially preferably 3. By appropriately selecting the types of substituents of R²⁵ to R²⁸, the absorption wavelength can be more reliably controlled. When the first compound is used as a p-type semiconductor material, the second compound being a compound represented by the general formula (4) serves as a n-type semiconductor material having good electron-transporting property when at least one of R²⁵ to R²⁸ is a group represented by the general formula (5).

The pyrene derivative is preferably a compound represented by the general formula (6).

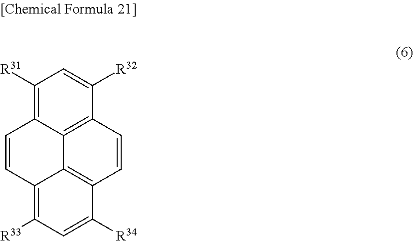

R³¹ to R³⁴ may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, an amino group, a silyl group, —P(═O)R³⁵R³⁶, and a group represented by the following general formula (5). R³⁵ and R³⁶ each represent an aryl group or a heteroaryl group. At least one of R³¹ to R³⁴ represents a group represented by the following general formula (5).

[Chemical Formula 22]

-LCN)ₙ (5)

In the general formula (5), n represents 1 or 2. L represents an alkenediyl group, an arylenediyl group or a heteroarylenediyl group when n represents 1. L represents an aklenetriyl group, an arylenetriyl group or a heteroarylenetriyl group when n represents 2.

The compound represented by the general formula (6) is a compound having a single peak absorption, and good color selectivity. By appropriately selecting the types of substituents of R³¹ to R³⁴, the absorption wavelength can be more reliably controlled. Particularly, it is preferable that at least one of R³¹ to R³⁴ is a group represented by the general formula (5) because the compound has an absorption region in a wavelength range of 400 to 700 nm, and serves as a n-type semiconductor material having good electron-transporting property.

The perylene derivative is preferably a compound represented by the general formula (7).

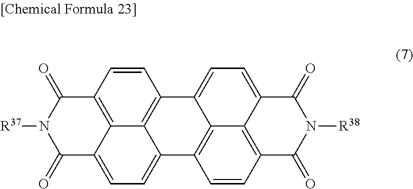

R³⁷ and R³⁸ may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, an amino group, a cyano group, a silyl group and —P(═O)R³⁹R⁴⁰. R³⁹ and R⁴⁰ each represent an aryl group or a heteroaryl group.

The compound represented by the general formula (7) is a compound having a high light absorption coefficient and good color selectivity. By appropriately setting the types of substituents of R³⁷ and R³⁸, the absorption wavelength can be controlled. The compound represented by the general formula (7) is preferably used as a n-type semiconductor because it has good electron-transporting property.

The absorption coefficient herein means a ratio of light absorbed per unit length when the light passes through a thin film. The absorption coefficient is a value calculated by substituting relevant values in the formula of (absorbency)/(thickness). Specifically, an organic compound is deposited with a thickness of 50 nm at a deposition rate of 1 Å/second on a 0.7 mm-thick transparent quartz glass using a vacuum vapor deposition method, an absorbency in a visible region of 400 nm to 700 nm is measured by an ultraviolet/visible spectrophotometer, and the maximum value of the absorbency is then divided by the thickness (unit: cm) of an organic compound to calculate an absorption coefficient.

The first compound represented by the general formula (1) can be used either as a p-type semiconductor material or as a n-type semiconductor material according to relative magnitudes of an ionization potential and electron affinity with respect to the second compound, but the first compound represented by the general formula (1) is preferably used as a p-type semiconductor material. The first compound represented by the general formula (1) is preferably used as a p-type semiconductor material particularly because the compound contains a group represented by the general formula (2) or (3), and is therefore excellent in hole transport property. The second compound is preferably a n-type semiconductor material.

The p-type semiconductor material as used herein represents a hole-transporting semiconductor material which has electron-donating property, and easily releases electrons (i.e. the p-type semiconductor material has a small ionization potential). The n-type semiconductor material represents an electron-transporting semiconductor material which has electron-accepting property, and easily accepts electrons (i.e. the n-type semiconductor material has high electron affinity). When the photoelectric conversion layer is composed of a p-type semiconductor material and a n-type semiconductor material, charges can be efficiently separated into holes and electrons before excitons produced by incident light in the photoelectric conversion layer returns to a ground state. Holes and electrons thus separated flow to the cathode and the anode through the p-type semiconductor material and the n-type semiconductor material, respectively, so that high photoelectric conversion efficiency can be achieved.

Electrodes and organic layers that form the photoelectric conversion element will now be described.

(Cathode and Anode)

In the photoelectric conversion element of the present invention, the cathode and the anode have a role of ensuring that electrons and holes produced in the photoelectric conversion element can flow to sufficiently feed a current. Preferably, at least one of the cathode and the anode is transparent or semitransparent for allowing light to be incident. Usually, a transparent electrode is used as the cathode to be formed on the substrate.

The cathode may be transparent for making it possible to extract holes from the photoelectric conversion layer and allowing light to be incident. When the cathode is a transparent electrode, the material of the cathode is preferably a conductive metal oxide such as tin oxide, indium oxide or indium tin oxide (ITO); a metal such as gold, silver or chromium; an inorganic conductive substance such as copper iodide or copper sulfide; a conductive polymer such as polythiophene, polypyrrole or polyaniline; or the like. When the cathode is used as a transparent electrode, it is especially preferable to use ITO glass with ITO on a grass substrate surface, or NESA glass with silver oxide on a glass substrate surface.

The transparent electrode may have resistance that allows a current produced in the photoelectric conversion element to sufficiently flow, and low resistance is preferable from the viewpoint of photoelectric conversion efficiency of the photoelectric conversion element. For example, an ITO substrate having a resistance of 300 WE or less serves as an element electrode, and therefore it is especially preferable to use a low-resistance product. The thickness of ITO or silver oxide can be appropriately selected according to the resistance value, and is commonly in a range of 50 to 300 nm. Soda-lime glass, alkali-free glass or the like is used for the glass substrate of ITO glass or NESA glass. The thickness of the glass substrate may have a thickness sufficient to maintain mechanical strength, and therefore a thickness of 0.5 mm or more is sufficient. The lesser ions eluted from the glass substrate, the better, so that the material of the glass substrate is preferably alkali-free glass, and soda-lime glass subjected to SiO₂ barrier coating can also be used. If the cathode stably functions, there is no need that the substrate be made of glass and, for example, a cathode may be formed on a plastic substrate. Examples of the method for formation of an ITO film include, but are not limited to, an electron beam method, a sputtering method, a chemical reaction method, and the like.

Preferably, the anode is made of a substance capable of efficiently extracting electrons from the photoelectric conversion layer, and examples thereof include platinum, gold, silver, copper, iron, tin, zinc, aluminum, indium, chromium, lithium, sodium, potassium, calcium, magnesium, cesium, and strontium. To improve element characteristics by enhancing electron extraction efficiency, lithium, sodium, potassium, calcium, magnesium, and cesium, or alloys containing these low work-function metals are effective. However, these low work-function metals are often unstable in the air in general, and it is possible to exemplify, as a preferable example, a method in which an electrode having high stability is used after doping the hole blocking layer with a trace amount of lithium, magnesium, or cesium (1 nm or less displayed by a film thickness meter of vacuum vapor deposition). It is also possible to use an inorganic salt such as lithium fluoride. To protect the electrode, it is preferred to laminate metals such as platinum, gold, silver, copper, iron, tin, aluminum, and indium, or alloys using these metals; inorganic substances such as silica, titania, and silicon nitride; polyvinyl alcohol, vinyl chloride, hydrocarbon-based polymers, and the like. The method for production of these electrodes is preferably a method capable of securing conduction, such as resistance heating, electron beam, sputtering, ion plating or coating.

When the photoelectric conversion element of the present invention is used as an image sensor, application of an electric field between the anode and the cathode from the outside produces an effect of improving photoelectric conversion efficiency because electrons and holes generated in the photoelectric conversion layer are easily guided to the anode side and the cathode side, respectively. Here, the applied voltage is preferably 10⁵ V/m or more and 10⁹ V/m or less. When the applied voltage is 10⁵ V/m or more, generated charges are easily transported efficiently, and therefore photoelectric conversion efficiency is hardly reduced. When the applied voltage is 10⁹ V/m or less, a dark current is reduced, so that the S/N ratio is improved, and the probability of occurrence of current leakage decreases. Even when an electric field is not applied between the anode and the cathode, charges are caused to flow to the photoelectric conversion element by an internal electric field at the time of connecting the anode and the cathode to make a closed circuit, and therefore it is also possible to use the photoelectric conversion element as a photovoltatic element.

(Photoelectric Conversion Layer)

The photoelectric conversion layer is a layer that causes photoelectric conversion in which incident light is absorbed to generate charges. The photoelectric conversion layer may be composed of one photoelectric conversion material, and is preferably composed of a p-type semiconductor material and a n-type semiconductor material. Here, the photoelectric conversion layer may include one or more p-type semiconductor material(s) and one or more n-type semiconductor material(s). In the photoelectric conversion layer, the photoelectric conversion material absorbs light to form excitons, and electrons and holes are then separated by the n-type semiconductor material and the p-type semiconductor material, respectively. Electrons and holes thus separated are caused to flow to both the electrodes through a conduction level and a valence level, so that electric energy is produced.

As a structure of the photoelectric conversion layer, a bulk heterojunction with the first compound and second compound mixed in the same layer by a method such as co-deposition is preferable. The bulk heterojunction is a structure in which two or more compounds are randomly mixed in one layer, and the compounds are joined together at a nano-level. Accordingly, charges generated in one of the materials can be efficiently separated into holes and electrons. For exhibiting high light-absorbing property, the absorption coefficient of the mixed film of the first compound and the second compound is preferably 5×10⁴ cm⁻¹ or more, more preferably 8×10⁴ cm⁻¹ or more, still more preferably 1×10⁵ cm⁻¹ or more.

Since the absorption coefficient of the whole thin film and the carrier-transporting property presented by the second compound are reduced as the mixing ratio of the first compound is increased, and the carrier property presented by the first compound is reduced as the mixing ratio of the second compound is increased, the mixing ratio of the first compound represented by the general formula (1) and the second compound (first compound:second compound) is preferably in a range of 75%:25% to 25%:75% in terms of a molar ratio. When the second compound having a large absorption coefficient is contained in a larger amount, the absorption coefficient of the whole thin film is improved, leading to improvement of photoelectric conversion efficiency, and therefore the mixing ratio of the first compound and the second compound (first compound:second compound) is more preferably in a range of 50%:50% to 25%:75%.

The first compound and the second compound are each required to have a function of efficiently transporting generated charges for obtaining high photoelectric conversion efficiency. The charge mobility of each of the first compound and the second compound is preferably 1×10⁻⁹ cm²/Vs or more, more preferably 1×10⁻⁸ cm²/Vs or more, still more preferably 1×10⁻⁷ cm²/Vs or more.

The charge mobility herein is mobility measured by a space charge limited current method (SCLC method) (see, for example, Adv. Funct. Mater, Vol.16 (2006), page 701 as a reference).

When the thickness of the organic layer is excessively small, the probability of occurrence of current leakage becomes higher, and the number of carriers generated decreases under the influence of thinning of the photoelectric conversion layer, so that photoelectric conversion efficiency is reduced. When the thickness of the organic layer is excessively large, carriers generated in the photoelectric conversion layer hardly arrive at the electrode, so that photoelectric conversion efficiency is reduced, and a higher electric field is required, leading to an increase in power consumption. Therefore, the thickness of the organic layer is preferably 20 nm or more and 200 nm or less.

For the photoelectric conversion material that forms the photoelectric conversion layer, a material previously known as a photoelectric conversion material may be used in combination in addition to the first compound and the second compound. When the first compound and the second compound are used in an organic layer other than the photoelectric conversion layer, materials previously known as photoelectric conversion materials can be used alone or in combination thereof.

The absorption wavelength of the photoelectric conversion layer is determined by the light absorption wavelength region of the photoelectric conversion material, so that it is preferable to use a material having light absorption characteristics corresponding to the color intended for use. For example, in the green photoelectric conversion element, the photoelectric conversion layer is composed of a material which absorbs light at a wavelength of 490 nm to 570 nm. When the photoelectric conversion layer is composed of two or more materials, and contain a p-type semiconductor material and a n-type semiconductor material, holes and electrons can be efficiently separated because among carriers generated in the photoelectric conversion layer, holes are apt to flow through the p-type semiconductor material, and electrons are apt to flow through the n-type semiconductor material. Therefore, for obtaining high photoelectric conversion efficiency, the photoelectric conversion layer is composed of a material including a p-type semiconductor material and a n-type semiconductor material which have mutually different energy levels, and the photoelectric conversion layer is composed of a material having high charge mobility so that holes and electrons generated in the photoelectric conversion layer can transfer to the electrode side.

The p-type semiconductor material may be any organic compound as long as it is a hole-transporting compound which has comparatively small ionization potential and has electron-donating property. Examples of the p-type organic semiconductor material include compounds including fused polycyclic aromatic derivatives such as naphthalene, anthracene, phenanthrene, pyrene, chrysene, naphthacene, triphenylene, perylene, fluoranthene, fluorene, indene, and derivatives thereof; cyclopentadiene derivatives, furan derivatives, thiophene derivatives, pyrrole derivatives, benzofuran derivatives, benzothiophene derivatives, indole derivatives, pyrazoline derivatives, dibenzofuran derivatives, dibenzothiophene derivatives, carbazole derivatives, indolocarbazole derivatives; aromatic amine derivatives such as
N,N′-dinaphthyl-N,N′-diphenyl-4,4′-diphenyl-1,1′-diamine; styrylamine derivatives, benzidine derivatives, porphyrin derivatives, phthalocyanine derivatives and quinacridone derivatives.

Examples of the polymer-based material include, but are not particularly limited to, polyphenylenevinylene derivatives, polyparaphenylene derivatives, polyfluorene derivatives, polyvinylcarbazole derivatives, and polythiophene derivatives.

The n-type semiconductor material may be any material as long as it is an electron-transporting compound having high electron affinity. Examples of the n-type semiconductor material include fused polycyclic aromatic derivatives such as naphthalene, anthracene and naphthacene; styryl-based aromatic ring derivatives typified by
4,4′-bis(diphenylethenyl)biphenyl, tetraphenylbutadiene derivatives, coumarin derivatives, oxadiazole derivatives, pyrrolopyridine derivatives, perinone derivatives, pyrrolopyrrole derivatives, thiadiazolopyridine derivatives, aromatic acetylene derivatives, aldazine derivatives, pyrromethene derivatives, diketopyrrolo[3,4-c]pyrrole derivatives; azole derivatives such as imidazole, thiazole, thiadiazole, oxazole, oxadiazole, and triazole, and metal complexes thereof; quinone derivatives such as anthraquinone and diphenoquinone; phosphorus oxide derivatives, and quinolinol complexes such as
tris(8-quinolinolate)aluminum(III); and various metal complexes such as benzoquinolinol complex, hydroxyazole complex, azomethine complex, tropolone-metal complex, and flavonol-metal complex.

Further, examples of the n-type semiconductor material include organic compounds having a nitro group, a cyano group, halogen, or a trifluoromethyl group in the molecule; acid anhydride-based compounds such as quinone-based compound, maleic anhydride, and phthalic anhydride; and fullerene and fullerene derivatives, such as C60 and PCBM.

Further, examples of the n-type semiconductor material include compounds having a heteroaryl ring structure which is composed of elements selected from hydrogen, nitrogen, oxygen, silicon and phosphorus, and contains electron-accepting nitrogen. The electron-accepting nitrogen as used herein represents a nitrogen atom which forms a multiple bond with an adjacent atom. Since a nitrogen atom has high electronegativity, the multiple bond has electron-accepting property. Therefore, an aromatic heterocyclic ring containing electron-accepting nitrogen has high electron affinity, and is thus preferable as a n-type semiconductor material.

Examples of the heteroaryl ring containing electron-accepting nitrogen include pyridine rings, pyrazine rings, pyrimidine rings, quinoline rings, quinoxaline rings, naphthyridine rings, pyrimidopyrimidine rings, benzoquinoline rings, phenanthroline rings, imidazole rings, oxazole rings, oxadiazole rings, triazole rings, thiadiazole rings, benzoxazole rings, benzothiazole rings, benzimidazole rings and phenanthroimidazole rings.

Examples of the preferred compound having such a heteroaryl ring structure include benzimidazole derivatives, benzoxazole derivatives, benzthiazole derivatives, oxadiazole derivatives, thiadiazole derivatives, triazole derivatives, pyrazine derivatives, phenanthroline derivatives, quinoxaline derivatives, quinoline derivatives, benzoquinoline derivatives, oligopyridine derivatives such as bipyridine and terpyridine, quinoxaline derivatives and naphthyridine derivatives. Among them, imidazole derivatives such as tris(N-phenylbenzimidazole-2-yl)benzene, oxadiazole derivatives such as
1,3-bis[(4-tert-butylphenyl)1,3,4-oxadiazolyl]phenylene, triazole derivatives such as
N-naphthyl-2,5-diphenyl-1,3,4-triazole, phenanthroline derivatives such as bathocuproin and
1,3-bis(1,10-phenanthroline-9-yl)benzene, benzoquinoline derivatives such as
2,2′-bis(benzo[h]quinoline-2-yl)-9,9′-spirobifluorene, bipyridine derivatives such as
2,5-bis(6′-(2′,2″-bipyridyl))-1,1-dimethyl-3,4-diphenylsilole, terpyridine derivatives such as
1,3-bis(4′-(2,2′:6′2″-terpyridinyl))benzene, naphthyridine derivatives such as
bis(1-naphthyl)-4-(1,8-naphthyridine-2-yl)phenylphosphine oxide, and the like are preferably used from the viewpoint of an electron-transporting ability.

Preferred n-type semiconductor materials that can be used include, but are not limited to, a group of the above-mentioned materials.

(Charge Blocking Layer)

The charge blocking layer is a layer for taking out electrons and holes photoelectrically converted by the photoelectric conversion layer in an efficient and stable manner, and examples thereof include an electron blocking layer for blocking electrons and a hole blocking layer for blocking holes. These layers may be composed of an inorganic substance or an organic compound. These layers may also be composed of a mixed layer of an inorganic substance and an organic compound.

The hole blocking layer is a layer for blocking recombination of holes produced in the photoelectric conversion layer with electrons as a result of flow of holes to the anode side. According to types of the material composing each layer, recombination of holes with electrons is suppressed by inserting this layer, leading to an improvement in photoelectric conversion efficiency. Thus, the hole blocking material preferably has an HOMO level which is energetically lower than that of the photoelectric conversion material. A compound capable of efficiently blocking transfer of holes from the photoelectric conversion layer is preferable, and specific examples thereof include quinolinol derivative metal complexes typified by 8-hydroxyquinoline aluminum; tropolone-metal complexes, flavonol-metal complexes, perylene derivatives, perinone derivatives, naphthalene derivatives, coumarin derivatives, oxadiazole derivatives, aldazine derivatives, bisstyryl derivatives, and pyrazine derivatives; oligopyridine derivatives such as bipyridine and terpyridine; phenanthroline derivatives, quinoline derivatives and aromatic phosphorus oxide compounds. These hole blocking materials may be used alone, or different hole blocking materials may be used in a state of being laminated or mixed.

The electron blocking layer is a layer for blocking recombination of electrons produced in the photoelectric conversion layer with holes as a result of flow of holes to the cathode side. According to types of the material composing each layer, recombination of holes with electrons is suppressed by inserting this layer, leading to an improvement in photoelectric conversion efficiency. Thus, the electron blocking material preferably has an LUMO level which is energetically higher than that of the photoelectric conversion material. A compound capable of efficiently blocking transfer of electrons from the photoelectric conversion layer is preferable, and specific examples thereof include triphenylamines such as
N,N′-diphenyl-N,N′-bis(3-methylphenyl)-4,4-diphenyl-1,1′-diamine and
N,N′-bis(1-naphthyl)-N,N′-diphenyl-4,4′-diphenyl-1,1′-diamine; bis(N-allylcarbazole) or bis(N-alkylcarbazole),
pyrazoline derivatives, stilbene-based compounds, distyryl derivatives, hydrazone-based compounds; and heterocyclic compounds typified by oxadiazole derivatives, phthalocyanine derivatives, and porphyrin derivatives. Examples of the polymer-based material include polycarbonate and styrene derivatives containing the monomer in the side chain, polyvinylcarbazole and polysilane. It is desirable to use a compound which forms a thin film required for production of the photoelectric conversion element and can extract holes from the photoelectric conversion layer, and also can transport holes. These electron blocking materials may be used alone, or different electron blocking materials may be used in a state of being laminated or mixed.

The above hole blocking layers and electron blocking layers can be used alone, or two or more of the materials can be used in a state of being laminated or mixed. It is also possible to use the hole blocking layer and the electron blocking layer in a state of being dispersed in solvent-soluble resins such as polyvinyl chloride, polycarbonate, polystyrene, poly(N-vinylcarbazole), polymethyl methacrylate, polybutyl methacrylate, polyester, polysulfone, polyphenylene oxide, polybutadiene, hydrocarbon resin, ketone resin, phenoxy resin, polysulfone, polyamide, ethyl cellulose, vinyl acetate, ABS resin, and polyurethane resin; and curable resins such as phenol resin, xylene resin, petroleum resin, urea resin, melamine resin, unsaturated polyester resin, alkyd resin, epoxy resin, and silicone resin; as a polymer binder.

Examples of the method for formation of an organic layer include, but are not limited to, a resistance heating vapor deposition method, an electron beam vapor deposition method, a sputtering method, a molecular lamination method, a coating method, and the like, and usually, the method is preferably a resistance heating vapor deposition method or an electron beam vapor deposition method in view of characteristics.

<Image Sensor>

The photoelectric conversion element of the present invention can be suitably used in an image sensor. The image sensor is a semiconductor element for converting an optical image into electrical signal. In general, the image sensor is composed of the above-mentioned photoelectric conversion element for converting light into electric energy, and a circuit for reading out electric energy in the form of electrical signal. According to applications of the image sensor, a plurality of photoelectric conversion elements can be aligned on one-dimensional straight line or two-dimensional plane. A monocolor image sensor may be composed of one photoelectric conversion element, but a color image sensor is composed of two or more photoelectric conversion elements. For example, the color image sensor is composed of a photoelectric conversion element for detecting red light, a photoelectric conversion element for detecting green light, and a photoelectric conversion element for detecting blue light. Photoelectric conversion elements of different colors have a laminated structure. Namely, the photoelectric conversion elements may be laminated on one pixel, or arranged side by side to form a matrix structure.

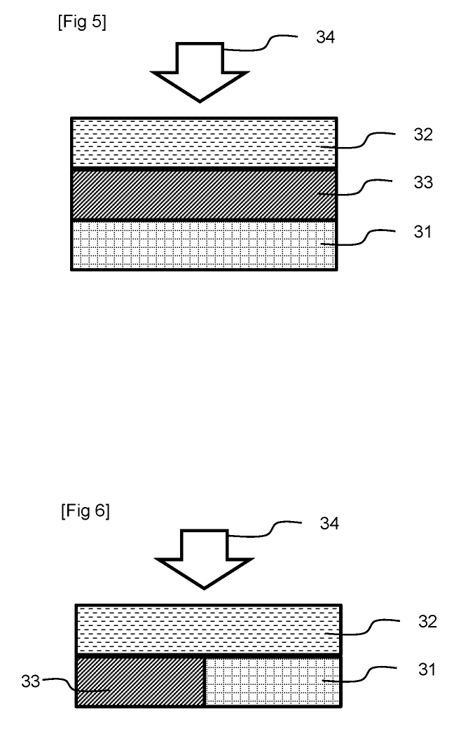

In the case of a structure in which a photoelectric conversion element is laminated on one pixel, as shown in FIG. 5, the structure may be a three-layer structure in which a photoelectric conversion element for detecting green light 32, a photoelectric conversion element for detecting blue light 33, and a photoelectric conversion element for detecting red light 31 are sequentially laminated. Alternatively, as shown in FIG. 6, the structure may be a two-layer structure in which a photoelectric conversion element for detecting green light 32 is disposed on the whole surface of an upper layer, and a photoelectric conversion element for detecting red light 31 and a photoelectric conversion element for detecting blue light 33 are formed in the form of a matrix structure. These structures are those in which the photoelectric conversion element 32 for detecting green light is disposed on a layer which is the nearest to incident light 34. The order of lamination of each color is not limited thereto, and may be different from that in FIG. 5. For ensuring that a photoelectric conversion element as an outer most layer serves as a color filter which absorbs light of specific color, and transmits long-wavelength light and short-wavelength light other than the light of specific color, a structure in which a green photoelectric conversion element is disposed as the outermost layer is preferable. When the blue photoelectric conversion element has excellent color selectivity, it is possible to use a structure in which the blue photoelectric conversion element is disposed as an uppermost layer from a viewpoint of ease of detecting a short wavelength.

In the case of a matrix structure, the array of photoelectric conversion elements can be selected from arrays such as Bayer array, honeycomb array, striped array, and delta array. An organic photoelectric conversion material is used in a photoelectric conversion element for detecting green light, and it is possible to appropriately use inorganic photoelectric conversion materials and organic photoelectric conversion materials, which have hitherto been used, in combination as for the photoelectric conversion element for detecting red light and the photoelectric conversion element for detecting blue light.

<Solar Cell>

The photoelectric conversion element of the present invention can be used in a solar cell. The solar cell is an energy conversion element which absorbs energy of sunlight and converts the energy of sunlight directly into electricity. The solar cell has a principle in common with an image sensor in that light is absorbed to generate electric energy, but the solar cell is different from the image sensor in that in the image sensor, charges generated in a photoelectric conversion layer are easily extracted by usually applying an electric field from the outside, whereas in the solar cell, a photoelectric conversion element itself generates photovoltatic power, so that charges generated in a photoelectric conversion layer are extracted to the outside.

The photoelectric conversion element of the present invention is suitable for conversion of light mainly in a visible region into electric energy because the photoelectric conversion element contains a compound that absorbs light having a wavelength of 400 to 700 nm. For improving the conversion efficiency of the solar cell, it is preferable to absorb light in a wavelength region that is as wide as possible. Therefore, a compound having light-absorbing property in the whole wavelength region of 400 to 700 nm is preferably used particularly as the second compound having a high light absorption coefficient. When the light absorption wavelength region is narrow in the photoelectric conversion element of the present invention, photoelectric conversion elements with mutually different light absorption wavelength regions (e.g. photoelectric conversion elements that absorb light of red, green and blue, respectively) may be laminated in a vertical form to produce a solar cell having a tandem structure.

<Single Color Detection Sensor>

The photoelectric conversion element of the present invention can be used in a single color detection sensor. The photoelectric conversion element can be suitably used in a single color detection sensor particularly when the photoelectric conversion element has color selectivity/color discriminability and a high light absorption coefficient. The single color detection sensor can be applied to remote controllers for televisions and electric appliances, light receiving elements for compact displayers, illuminance sensors, fluorescent probe sensors, CCDs, photoresistors and so on, but the application of the single color detection sensor is not limited thereto.

<Flexible Sensor>

The photoelectric conversion element of the present invention can be used in a flexible sensor. A photoelectric conversion element using an organic compound is lighter and more flexible than an existing photoelectric conversion element using an inorganic semiconductor. By making use of this feature, the photoelectric conversion element can be mounted on a curved-surface structure, or mounted for imaging a surface of a living body. Since the photoelectric conversion element can be produced in a printing process, a sensor with a large area can be produced.

### EXAMPLES

The present invention will be described below by way of Examples, but the present invention is not limited to these Examples. The number of each compound in the following Examples indicates the number of each of the foregoing compounds. Evaluation methods with respect to structural analysis are shown below.

Using superconductive FTNMR EX-270 (manufactured by JEOL, Ltd.), ¹H-NMR was measured by a deuterated chloroform solution.

Using a U-3200 type spectrophotometer (manufactured by Hitachi, Ltd.), an absorption spectrum was measured after vapor deposition of a sample in a film thickness of 50 nm on a quartz substrate. An absorption coefficient was calculated by Lambert-Beer Law.

The spectral sensitivity characteristics (external quantum efficiency and maximum sensitivity wavelength) of a photoelectric conversion element were measured using a spectral sensitivity measurement system Model SM-250 (manufactured by Bunkoukeiki Co., Ltd.).

### Synthesis Example 1

Method for Synthesis of Compound [10]

A mixed solution of phenylacetylene (10.0 g) and dehydrated tetrahydrofuran (200 ml) was stirred at 0° C. under a nitrogen gas flow. To this mixed solution was added dropwise n-butyllithium (1.6 M hexane solution (62 ml)), and the mixture was then stirred at 0° C. for 2 hours. Thereafter, a mixed solution of phenylacetaldehyde (6.0 g) and dehydrated tetrahydrofuran (20 ml) was added dropwise, and the mixture was then returned to room temperature, and stirred for 4 hours. To the reaction solution was added pure water (100 ml), and the mixture was then extracted with ethyl acetate. The thus obtained solution was dried over magnesium sulfate and, after filtration, the solvent was distilled off. The thus obtained liquid was purified by silica gel column chromatography, and evaporated to obtain a yellow liquid (9.0 g).

Next, a mixed solution of the yellow liquid (9. 0 g), sodium bicarbonate (6.8 g), iodine (30.8 g) and acetonitrile (400 ml) was stirred at room temperature for 4 hours under a nitrogen flow. To the reaction solution was added a saturated sodium thiosulfate aqueous solution (100 ml), and the mixture was stirred for 1 hour, and then extracted with ethyl acetate. The thus obtained solution was dried over magnesium sulfate and, after filtration, the solvent was distilled off. The thus obtained liquid was purified by silica gel column chromatography, and evaporated to obtain a yellow liquid (9.3 g)

Next, a mixed solution of the yellow liquid (9.3 g) and dehydrated tetrahydrofuran (56 ml) was stirred at −78° C. under a nitrogen flow. To this mixed solution was added dropwise n-butyllithium (1.6 M hexane solution (19 ml)), and the mixture was then stirred at −78° C. for 2 hours. To the reaction solution was added 5,12-naphthacenequinone (2.9 g) for 30 minutes, and the mixture was then stirred at room temperature for 4 hours. To the reaction solution was added pure water (100 ml), and the mixture was evaporated to remove a half of the tetrahydrofuran, and then extracted with dichloromethane. The thus obtained solution was dried over magnesium sulfate and, after filtration, the solvent was distilled off. The thus obtained solid was dissolved in a small amount of dichloromethane, and then precipitated by adding methanol, and the mixture was filtered.

The thus obtained solid was vacuum-dried to obtain a yellow powder (2.8 g).

Next, a mixed solution of the yellow powder (2.8 g) and dehydrated tetrahydrofuran (43 ml) was stirred at 40° C. under a nitrogen flow. To this mixed solution were added dropwise concentrated hydrochloric acid (22.4 ml) and tin chloride (II) dihydrate (9.6 g), and the mixture was then stirred for 4 hours. The reaction solution was returned to room temperature, methanol (100 ml) was then added, and the mixture was stirred for 30 minutes, and then filtered. The thus obtained solid was washed with pure water and methanol, and then filtered. The thus obtained solid was purified by silica gel column chromatography, and evaporated to obtain an orange powder (550 mg).

The results of ¹H-NMR analysis of the thus-obtained powder are as follows, and show that the thus obtained orange powder is a compound [10].

¹H-NMR (CDCl₃ (d=ppm)): 6.70-7.74 (m, 26H), 8.04-9.09 (t, 4H), 8.19 (s, 2H).

Light absorption characteristics of the compound [10] are as follows.
Maximum absorption wavelength: 504 nm (thin film: 50 nm)
Full width at half maximum at maximum absorption wavelength: 23 nm
Absorption coefficient at maximum absorption wavelength: 4.72×10⁴ cm⁻¹

### Synthesis Example 2

Method for Synthesis of Compound [43]

A mixed solution of 2-bromoacetophenone (35.0 g), phenol (18.2 g), potassium carbonate (26.7 g) and acetone (700 ml) was refluxed for 5 hours under a nitrogen flow. The reaction solution was returned to room temperature, evaporated to remove the solvent, and then extracted with toluene. The thus obtained solution was dried over magnesium sulfate, and then evaporated to remove the solvent. The thus obtained solid was recrystallized with methanol to obtain a white powder (23.0 g).

Next, a mixed solution of the white powder (23.0 g), methanesulfonic acid (52.0 g) and toluene (430 ml) was stirred at 80° C. for 6 hours under a nitrogen flow. The reaction solution was returned to room temperature, pure water (400 ml) was added, and the mixture was stirred for 30 minutes, and then extracted with toluene. The thus obtained solution was dried over magnesium sulfate, and then evaporated to remove the solvent. The thus obtained solution was purified by silica gel column chromatography, and evaporated to obtain a colorless liquid (19.0 g).

Next, a mixed solution of the colorless liquid (19.0 g) and dehydrated tetrahydrofuran (200 ml) was stirred at 0° C. under a nitrogen flow. To this mixed solution was added dropwise n-butyllithium (1.6 M hexane solution (61 ml)), and the mixture was then stirred at 0° C. for 3 hours. To the reaction solution was added 5,12-naphthacenequinone (10.1 g) for 30 minutes, and the mixture was then stirred at 0° C. for 1 hour. The reaction solution was returned to room temperature, and further stirred for 1 hour, pure water (200 ml) and toluene (200 ml) were then added, and the mixture was stirred for 30 minutes. The organic layer was separated, and then dried over magnesium sulfate, and evaporated to remove the solvent. The thus obtained solid was recrystallized with toluene to obtain a white powder (21.4 g).

Next, a mixed solution of the white powder (21.4 g), sodium hypophosphite monohydrate (34.9 g), potassium iodide (36.2 g) and acetic acid (330 ml) was refluxed for 2 hours under a nitrogen flow. To the reaction solution was added pure water (350 ml), and the mixture was stirred for 30 minutes, and then filtered. To the thus obtained solid was added cyclopentyl methyl ether (200 ml), and the mixture was refluxed for 2 hours, and then filtered. The thus obtained solid was purified by silica gel column chromatography, and evaporated to obtain an orange powder (15.5 g).

The results of ¹H-NMR analysis of the thus-obtained powder are as follows, and show that the thus obtained orange powder is a compound [43].

¹H-NMR (CDCl₃ (d=ppm)): 7.06-8.29 (m, 26H), 8.50 (s, 2H)

Light absorption characteristics of the compound [43] are as follows.
Maximum absorption wavelength: 512 nm (thin film: 50 nm)
Full width at half maximum at maximum absorption wavelength: 103 nm
Absorption coefficient at maximum absorption wavelength: 2.75×10⁴ cm⁻¹

### Synthesis Example 3

Method for Synthesis of Compound [108]

A mixed solution of 1-bromomethyl-2-dibromomethylnaphthalene (10.0 g), 1,4-naphthoquinone (5.2 g), sodium iodide (25.5 g) and dehydrated dimethylformamide (85 ml) was stirred at 70° C. for 6 hours under a nitrogen flow. The reaction solution was returned to room temperature, and then filtered. The thus obtained solid was washed with pure water and methanol, and then filtered. The thus obtained solid was vacuum-dried to obtain a yellow powder (4.32 g).

Next, a mixed solution of 3-phenylbenzofuran (5.9 g) and dehydrated tetrahydrofuran (50 ml) was stirred at 0° C. under a nitrogen gas flow. To this mixed solution was added dropwise n-butyllithium (1.6 M hexane solution (15 ml)), and the mixture was then stirred at 0° C. for 3 hours. To the reaction solution was added the yellow powder (3.0 g) for 30 minutes, and the mixture was then stirred at 0° C. for 1 hour. The reaction solution was returned to room temperature, and further stirred for 1 hour, pure water (100 ml) and toluene (100 ml) were then added, and the mixture was stirred for 30 minutes. The organic layer was separated, and then dried over magnesium sulfate, and evaporated to remove the solvent. The thus obtained solid was recrystallized with toluene to obtain a white powder (5.4 g).

Next, a mixed solution of the white powder (5.4 g), sodium hypophosphite monohydrate (8.2 g), potassium iodide (8.5 g) and acetic acid (80 ml) was refluxed for 2 hours under a nitrogen flow. To the reaction solution was added pure water (80 ml), and the mixture was stirred for 30 minutes, and then filtered. To the thus obtained solid was added cyclopentyl methyl ether (50 ml), and the mixture was refluxed for 2 hours, and then filtered. The thus obtained solid was vacuum-dried to obtain a yellow powder (2.7 g).

The results of ¹H-NMR analysis of the thus-obtained powder are as follows, and show that the thus obtained orange powder is a compound [108].

¹H-NMR (CDCl₃ (d=ppm))): 7.08-7.13 (m, 7H), 7.25-7.51 (m, 13H), 7.69-7.75 (m, 3H), 7.89-7.96 (m, 2H), 8.04-8.08 (m, 2H), 8.34-8.35 (m, 2H), 9.19-9.22 (d, 1H, d=7.56Hz)

Light absorption characteristics of the compound [108] are as follows.
Maximum absorption wavelength: 492 nm (thin film: 50 nm)
Full width at half maximum at maximum absorption wavelength:

absorption spectrum has no clear peak, and calculation is impossible.
Absorption coefficient at maximum absorption wavelength: 3.00×10⁻⁴ cm⁻¹

### Synthesis Example 4

Method for Synthesis of Compound [7]

To 2,4-diphenylamine (24.5 g) was added 3 N aqueous hydrochloric acid (300 mL) in an argon atmosphere, and the mixture was heated to 60° C. in an oil bath, and stirred for 4 hours to form a hydrochloric acid salt (white suspension liquid). The white suspension liquid was cooled to 5° C. or lower in a sodium chloride-ice bath, and an aqueous solution (60 mL) containing sodium nitrite (8.27 g) was added dropwise for 30 minutes under stirring. Here, the liquid temperature was adjusted so as not to exceed 10° C. The produced reddish-brown solution was further stirred at 5° C. for 1 hour to prepare a diazonium salt solution. An aqueous solution (180 mL) containing potassium iodide (60 g) was prepared in a beaker, and the prepared diazonium salt solution was gradually added for 30 minutes under stirring. The mixture was further stirred for 30 minutes until a nitrogen gas was no longer generated, and methylene chloride (200 mL) was then added to dissolve the product. A small amount of sodium hydrogen sulfite was added to decompose iodine generated as a byproduct, and the organic layer was then separated, washed with aqueous sodium carbonate and water, and then dried over magnesium sulfate. The solvent was distilled off under reduced pressure, and the organic layer was purified by column chromatography to obtain 2,4-diphenyl benzene iodide (29.4 g) (yield: 82.5%).

2,4-diphenyl benzene iodide (27.4 g) was dissolved in dehydrated toluene (180 mL) and dehydrated ether (60 mL) in an argon atmosphere, and the solution was cooled to −45° C. in a dry ice-acetone bath. To this was added dropwise a 2.44 M n-butyllithium-n-hexane solution (31 mL) for 15 minutes, the temperature was slowly lowered to −10° C., and the mixture was stirred for 1 hour. To this was added 5, 12-naphthoquinone (7.75 g) little by little for 30 minutes, the temperature was then gradually elevated to room temperature, and the mixture was further stirred for 5 hours. The mixture was cooled to 0° C. with iced water, and methanol (60 mL) was added dropwise. The produced powder was collected by filtration, washed with cold methanol several times, and vacuum-dried to obtain a white powder. Toluene (200 mL) was added, and the mixture was heated and suspended for 1 hour, and cooled to room temperature. The mixture was filtered, washed with cold toluene, and vacuum-dried to obtain a white powder of a diol (15.1 g) (yield: 69.8%).

The following reaction was carried out while a flask provided with an argon blowing tube was shielded against light with an aluminum foil. To the diol (14.42 g) was added degassed tetrahydrofuran (THF) (450 mL), and the diol was dissolved by stirring the mixture while blowing argon. Therefore, the solution was heated to 40° C. in an oil bath. To this was added dropwise a concentrated hydrochloric acid aqueous solution (150 mL) containing tin dichlorate dihydrate (45.1 g) for 90 minutes. Thereafter, the temperature of the oil bath was elevated to 70° C., and the mixture was stirred under reflux for 2 hours, and cooled to room temperature. A 2 L beaker was shielded against light, distilled water (1 L) was added therein, and degassing was performed by feeding an argon flow. The reaction liquid was added therein, and the mixture was stirred for 30 minutes. The precipitated yellow powder was collected by filtration, and added in distilled water (1 L) again, and the mixture was stirred and washed. The mixture was filtered, sufficiently washed with methanol, and then vacuum-dried. This was heated and suspended in acetone (250 mL) degassed by blowing argon, and the suspension was filtered and vacuum-dried to obtain an orange-yellow powder of a desired compound [7] (12.70 g) (yield: 92.7%).

Optical characteristics of the compound [7] are as follows.
Maximum absorption wavelength: 506 nm (thin film: 50 nm)
Full width at half maximum at maximum absorption wavelength: 23 nm
Absorption coefficient at maximum absorption wavelength: 4.65×10⁴ cm

### Example 1

A photoelectric conversion element using the compound [10] was produced in the following manner. A glass substrate (manufactured by Asahi Glass Co., Ltd., 15 Ω/□, electron beam vapor-deposited product) including an ITO transparent conductive film having a film thickness of 150 nm deposited thereon was cut into pieces of 30×40 mm in size, followed by etching. The substrate thus obtained was subjected to ultrasonic cleaning for 15 minutes each, using acetone and “SEMICOCLEAN (registered trademark) 56” (manufactured by Furuuchi Chemical Corporation), and then washed with ultra-pure water. Subsequently, the substrate was subjected to ultrasonic cleaning with isopropyl alcohol for 15 minutes, then immersed in hot methanol for 15 minutes, and dried. Immediately before the production of a photoelectric conversion element, this substrate was subjected to a UV ozone treatment for 1 hour. After placing in a vacuum vapor deposition device, the inside of the device was evacuated until the degree of vacuum became 5×10⁻⁵ Pa or less. Molybdenum oxide was vapor-deposited as an electron blocking layer in a film thickness of 30 nm by a resistance heating method. Next, as a photoelectric conversion layer, the compound [10] being a p-type semiconductor material and the compound A-1 being a n-type semiconductor material were co-deposited in a film thickness of 70 nm at a vapor deposition rate ratio of 1:3. Next, aluminum was vapor-deposited as a cathode in a film thickness of 60 nm to produce a photoelectric conversion element of 2×2 mm square. The film thickness as used herein is an indicated value of a crystal oscillation type thickness monitor.

For production of a substrate for absorption spectrum measurement, a quartz substrate was placed in the same chamber concurrently with vapor deposition of the photoelectric conversion layer, so that a 70 nm-thick thin film was formed on the quartz substrate.

The absorption spectrum of vapor-deposited film on the quartz substrate at 400 nm to 700 nm was measured using an ultraviolet/visible spectrophotometer. Light absorption characteristics are as follows.
Maximum absorption wavelength: 525 nm
Full width at half maximum at maximum absorption wavelength: 143 nm
Absorption coefficient at maximum absorption wavelength: 9.88×10 ⁴ cm⁻¹

Spectral sensitivity characteristics in application of a bias voltage (−3 V) to the photoelectric conversion element are as follows.
Maximum sensitivity wavelength: 540 nm
External quantum efficiency at maximum sensitivity wavelength: 50%

In the present invention, photoelectric conversion efficiency is evaluated by external quantum efficiency at the maximum sensitivity wavelength.

### Examples 2 to 9

Except that the types of a p-type semiconductor material and a n-type semiconductor material, and the vapor deposition rate ratio were set as shown in Table 1, the same procedure as in Example 1 was carried out to produce a photoelectric conversion element. Light absorption characteristics and spectral sensitivity characteristics are shown in Table 1.

TABLE 1

| | | | | | | | | | External quantum | |
|---|---|---|---|---|---|---|---|---|---|---|
| | | | | | | | | | efficiency at | |
| | | | | | | | Absorption | | maximum | |
| | | | | | | Full | coefficient | | sensitivity | |
| | | p-type | n-type | Vapor | Maximum | width | at maximum | Maximum | wavelength (%) | |
| | Electron | semi- | semi- | deposition | absorption | at half | absorption | sensitivity | Voltage | Voltage |
| | blocking | conductor | conductor | rate ratio | wavelength | maximum | wavelength | wavelength | applied: | applied: |
| | layer | material | material | (p-type:n-type) | (nm) | (nm) | (cm⁻¹) | (nm) | none | −3 V |
| Example 1 | MoO₃ | Compound | A-1 | 1:3 | 525 | 143 | 9.88 × 10⁴ | 540 | 18 | 50 |
| | | [10] | | | | | | | | |
| Example 2 | MoO₃ | Compound | A-1 | 1:1 | 525 | 140 | 8.90 × 10⁴ | 530 | 14 | 39 |
| | | [10] | | | | | | | | |
| Example 3 | MoO₃ | Compound | A-1 | 3:1 | 505 | 139 | 3.09 × 10⁴ | 510 | 10 | 34 |
| | | [10] | | | | | | | | |
| Example 4 | MoO₃ | Compound | A-1 | 1:3 | 526 | 149 | 9.34 × 10⁴ | 540 | 10 | 43 |
| | | [43] | | | | | | | | |
| Example 5 | MoO₃ | Compound | A-1 | 1:1 | 525 | 144 | 7.44 × 10⁴ | 520 | 3 | 32 |
| | | [43] | | | | | | | | |
| Example 6 | MoO₃ | Compound | A-1 | 3:1 | 505 | 139 | 3.09 × 10⁴ | 500 | 1 | 11 |
| | | [43] | | | | | | | | |
| Example 7 | MoO₃ | Compound | A-1 | 1:3 | 524 | 137 | 9.11 × 10⁴ | 530 | 3 | 35 |
| | | [108] | | | | | | | | |
| Example 8 | MoO₃ | Compound | A-1 | 1:1 | 524 | 143 | 9.88 × 10⁴ | 540 | 5 | 36 |
| | | [108] | | | | | | | | |
| Example 9 | MoO₃ | Compound | A-1 | 3:1 | 500 | 148 | 3.50 × 10⁴ | 490 | 1 | 12 |
| | | [108] | | | | | | | | |

### Examples 10 to 30

Except that as an electron blocking layer, PEDOT/PSS (Clevios™ P VP A14083) was applied in a film thickness of 30 nm instead of vapor-depositing molybdenum oxide in a film thickness of 30 nm, and the types of a p-type semiconductor material and a n-type semiconductor material, and the vapor deposition rate ratio were set as shown in Table 2, the same procedure as in Example 1 was carried out to produce a photoelectric conversion element. Light absorption characteristics and spectral sensitivity characteristics are shown in Table 2.

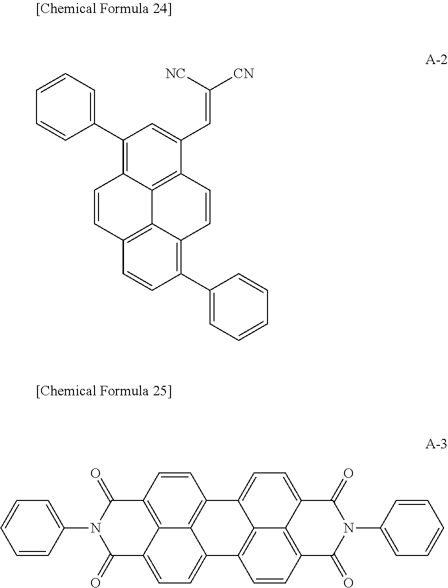 [SMILES: N#CC(C#N)=Cc1cc(-c2ccccc2)c2ccc3ccc(-c4ccccc4)c4ccc1c2c34 ; O=C1c2ccc3c4ccc5c6c(ccc(c7ccc(c2c37)C(=O)N1c1ccccc1)c64)C(=O)N(c1ccccc1)C5=O]

TABLE 2

| | | | | | | | | | External quantum | |
|---|---|---|---|---|---|---|---|---|---|---|
| | | | | | | | | | efficiency at | |
| | | | | | | | Absorption | | maximum | |
| | | | | | | Full | coefficient | | sensitivity | |
| | | p-type | n-type | Vapor | Maximum | width | at maximum | Maximum | wavelength (%) | |
| | Electron | semi- | semi- | deposition | absorption | at half | absorption | sensitivity | Voltage | Voltage |
| | blocking | conductor | conductor | rate ratio | wavelength | maximum | wavelength | wavelength | applied: | applied: |
| | layer | material | material | (p-type:n-type) | (nm) | (nm) | (cm⁻¹) | (nm) | none | −3 V |
| Example 10 | PEDOT: PSS | Compound | A-1 | 1:3 | 525 | 143 | 9.88 × 10⁴ | 510 | 18 | 53 |
| | | [10] | | | | | | | | |
| Example 11 | PEDOT: PSS | Compound | A-1 | 1:1 | 525 | 140 | 8.90 × 10⁴ | 530 | 27 | 64 |
| | | [10] | | | | | | | | |
| Example 12 | PEDOT: PSS | Compound | A-1 | 3:1 | 505 | 139 | 3.09 × 10⁴ | 540 | 14 | 47 |
| | | [10] | | | | | | | | |
| Example 13 | PEDOT: PSS | Compound | A-1 | 1:3 | 526 | 149 | 9.34 × 10⁴ | 540 | 4 | 25 |
| | | [43] | | | | | | | | |
| Example 14 | PEDOT: PSS | Compound | A-1 | 1:1 | 525 | 144 | 7.44 × 10⁴ | 520 | 5 | 34 |
| | | [43] | | | | | | | | |
| Example 15 | PEDOT: PSS | Compound | A-1 | 3:1 | 505 | 139 | 3.09 × 10⁴ | 510 | 3 | 25 |
| | | [43] | | | | | | | | |
| Example 16 | PEDOT: PSS | Compound | A-1 | 1:3 | 524 | 137 | 9.11 × 10⁴ | 530 | 5 | 36 |
| | | [108] | | | | | | | | |
| Example 17 | PEDOT: PSS | Compound | A-1 | 1:1 | 524 | 143 | 9.88 × 10⁴ | 540 | 7 | 37 |
| | | [108] | | | | | | | | |
| Example 18 | PEDOT: PSS | Compound | A-1 | 3:1 | 500 | 148 | 3.50 × 10⁴ | 490 | 1 | 14 |
| | | [108] | | | | | | | | |
| Example 19 | PEDOT: PSS | Compound | A-1 | 1:1 | 522 | 140 | 9.64 × 10⁴ | 520 | 16 | 52 |
| | | [7] | | | | | | | | |
| Example 20 | PEDOT: PSS | Compound | A-2 | 1:3 | 477 | 90 | 5.66 × 10⁴ | 480 | 6 | 38 |
| | | [10] | | | | | | | | |
| Example 21 | PEDOT: PSS | Compound | A-2 | 1:1 | 479 | 90 | 4.37 × 10⁴ | 470 | 7 | 36 |
| | | [10] | | | | | | | | |
| Example 22 | PEDOT: PSS | Compound | A-2 | 3:1 | 473 | 97 | 2.15 × 10⁴ | 470 | 2 | 20 |
| | | [10] | | | | | | | | |
| Example 23 | PEDOT: PSS | Compound | A-2 | 1:3 | 481 | 93 | 4.68 × 10⁴ | 490 | 1 | 14 |
| | | [43] | | | | | | | | |
| Example 24 | PEDOT: PSS | Compound | A-2 | 1:1 | 481 | 93 | 4.56 × 10⁴ | 480 | 1 | 15 |
| | | [43] | | | | | | | | |
| Example 25 | PEDOT: PSS | Compound | A-3 | 1:3 | 502 | 110 | 5.99 × 10⁴ | 480 | 4 | 47 |
| | | [10] | | | | | | | | |
| Example 26 | PEDOT: PSS | Compound | A-3 | 1:1 | 503 | 77 | 4.41 × 10⁴ | 480 | 11 | 43 |
| | | [10] | | | | | | | | |
| Example 27 | PEDOT: PSS | Compound | A-3 | 3:1 | 503 | 23 | 5.16 × 10⁴ | 470 | 3 | 17 |
| | | [10] | | | | | | | | |
| Example 28 | PEDOT: PSS | Compound | A-3 | 1:3 | 508 | 100 | 5.94 × 10⁴ | 490 | 4 | 38 |
| | | [43] | | | | | | | | |
| Example 29 | PEDOT: PSS | Compound | A-3 | 1:1 | 501 | 106 | 4.78 × 10⁴ | 490 | 6 | 38 |
| | | [43] | | | | | | | | |
| Example 30 | PEDOT: PSS | Compound | A-3 | 3:1 | 499 | 110 | 3.64 × 10⁴ | 500 | 1 | 10 |
| | | [43] | | | | | | | | |

### Comparative Examples 1 to 7

Except that only one of a n-type semiconductor material and a p-type semiconductor material was used in a photoelectric conversion layer, the same procedure as in Example 1 was carried out to produce a photoelectric conversion element. Light absorption characteristics and spectral sensitivity characteristics are shown in Table 3.

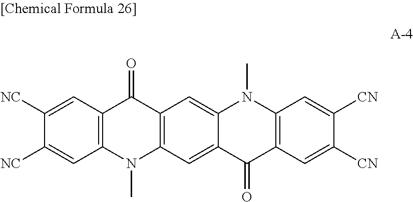 [SMILES: Cn1c2cc(C#N)c(C#N)cc2c(=O)c2cc3c(cc21)c(=O)c1cc(C#N)c(C#N)cc1n3C]

TABLE 3

| | | | | | | | | | External quantum | |
|---|---|---|---|---|---|---|---|---|---|---|
| | | | | | | | | | efficiency at | |
| | | | | | | | Absorption | | maximum | |
| | | | | | | Full | coefficient | | sensitivity | |
| | | p-type | n-type | Vapor | Maximum | width | at maximum | Maximum | wavelength (%) | |
| | Electron | semi- | semi- | deposition | absorption | at half | absorption | sensitivity | Voltage | Voltage |
| | blocking | conductor | conductor | rate ratio | wavelength | maximum | wavelength | wavelength | applied: | applied: |
| | layer | material | material | (p-type:n-type) | (nm) | (nm) | (cm⁻¹) | (nm) | none | −3 V |
| Comparative | MoO₃ | Compound | | | 504 | 23 | 4.72 × 10⁴ | | 0 | 0 |
| Example 1 | | [10] | | | | | | | | |
| Comparative | MoO₃ | Compound | | | 512 | 103 | 2.75 × 10⁴ | | 0 | 0 |
| Example 2 | | [43] | | | | | | | | |
| Comparative | MoO₃ | Compound | | | 492 | None | 3.00 × 10⁴ | | 0 | 0 |
| Example 3 | | [108] | | | | | | | | |
| Comparative | MoO₃ | | A-1 | | 533 | 163 | 1.16 × 10⁵ | 570 | 0 | 4 |
| Example 4 | | | | | | | | | | |
| Comparative | MoO₃ | | A-2 | | 489 | 105 | 6.51 × 10⁴ | | 0 | 0 |
| Example 5 | | | | | | | | | | |
| Comparative | MoO₃ | | A-3 | | 500 | 113 | 8.65 × 10⁴ | | 0 | 0 |
| Example 6 | | | | | | | | | | |
| Comparative | MoO₃ | | A-4 | | 547 | 70 | 2.34 × 10⁴ | | 0 | 0 |
| Example 7 | | | | | | | | | | |
| Comparative | MoO₃ | Compound | A-4 | 1:1 | 516 | 83 | 2.97 × 10⁴ | 520 | 0 | 1 |
| Example 8 | | [10] | | | | | | | | |
| Comparative | MoO₃ | Compound | A-4 | 1:1 | 504 | 45 | 2.90 × 10⁴ | 500 | 0 | 1 |
| Example 9 | | [43] | | | | | | | | |
| Comparative | MoO₃ | Compound | A-4 | 1:1 | 496 | 57 | 2.69 × 10⁴ | 500 | 0 | 1 |
| Example 10 | | [108] | | | | | | | | |

### Comparative Example 8

Except that a compound A-4 was used as a n-type semiconductor material, the same procedure as in Example 1 was carried out to produce a photoelectric conversion element. Light absorption characteristics and spectral sensitivity characteristics are shown in Table 3.

### Comparative Examples 9 and 10

Except that as a p-type semiconductor material, one as shown in Table 3 was used, the same procedure as in Comparative Example 7 was carried out to produce a photoelectric conversion element. Light absorption characteristics and spectral sensitivity characteristics are shown in Table 3.

### INDUSTRIAL APPLICABILITY

The photoelectric conversion element of the present invention can be applied in the fields of image sensors and solar cells. Specifically, the photoelectric conversion element can be employed in the fields of image elements mounted in mobile phones, smartphones, tablet PCs, digital still cameras, and the like; and sensing devices such as photovoltaic power generating apparatuses and visible light sensors.

### DESCRIPTION OF REFERENCE SIGNS

10: First electrode

11: Organic layer

13: Electron blocking layer

15: Photoelectric conversion layer

17: Hole blocking layer

20: Second electrode

31: Photoelectric conversion element for detecting red light

32: Photoelectric conversion element for detecting green light

33: Photoelectric conversion element for detecting blue light

34: Incident light

1. A photoelectric conversion element including a first electrode, a second electrode, and at least one organic layer existing therebetween, the organic layer containing a first compound, and a second compound in which the maximum value of an absorption coefficient at a wavelength of 400 to 700 nm is 5×10⁴ cm⁻¹ or more, the first compound being represented by the following general formula (1): 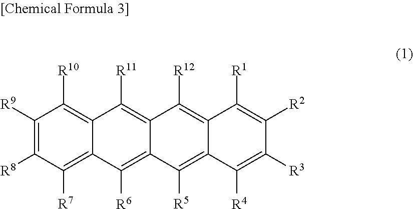
(in the general formula (1), R¹ to R¹² may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, a carbonyl group, a carboxyl group, an oxycarbonyl group, a carbamoyl group, an amino group, a nitro group, a cyano group, a silyl group and —P(═O)R¹³R¹⁴; R¹³ _{and R}¹⁴ each represent an aryl group or a heteroaryl group; adjacent substituents may be linked together to form a ring structure; and
R⁵ and R¹² in the general formula (1) each represent a group represented by the following general formula (2) or (3): 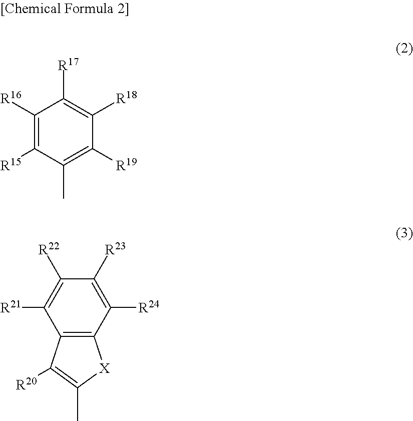
(in the general formula (2) or (3), R¹⁵ to R²⁴ may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, a carbonyl group, a carboxyl group, an oxycarbonyl group, a carbamoyl group, an amino group, a nitro group, a cyano group, a silyl group and —P(═O)R¹³R¹⁴; R¹³ and R¹⁴ each represent an aryl group or a heteroaryl group; R¹⁶ to R¹⁹ and R²¹ to R²⁴ may form a ring between adjacent substituents; X represents an oxygen atom, a sulfur atom or —NR²⁵; and R²⁵ is hydrogen, an alkyl group, a cycloalkyl group, a heterocyclic group, an aryl group or a heteroaryl group)).

2. The photoelectric conversion element according to claim 1, wherein R⁵ and R¹² in the general formula (1) are each represented by the general formula (2), and R¹⁵ in the general formula (2) represents an alkyl group, an alkoxy group, an aryl group or a heteroaryl group.

3. The photoelectric conversion element according to claim 1, wherein R⁵ and R¹² in the general formula (1) are each represented by the general formula (3), and R²⁹ in the general formula (3) represents an alkyl group, an alkoxy group, an aryl group or a heteroaryl group.

4. The photoelectric conversion element according to claim 1, wherein R⁵ and R¹² in the general formula (1) are each represented by the general formula (3), and X in the general formula (3) represents an oxygen atom.

5. The photoelectric conversion element according to claim 1, wherein the second compound is a derivative selected from a thiophene derivative, a pyrene derivative and a perylene derivative.

6. The photoelectric conversion element according to claim 5, wherein the second compound is a compound represented by the following general formula (4): 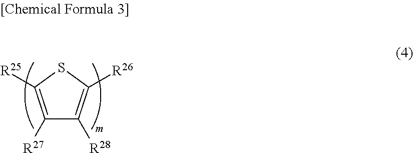
(in the general formula (4), R²⁵ to R²⁸ may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, an amino group, a silyl group, —P(═O)R²⁹R³⁰, and a group represented by the following general formula (5); R²⁹ and R³⁰ each represent an aryl group or a heteroaryl group; m represents an integer of 1 to 6; and at least one of R²⁵ to R²⁸ represents a group represented by the following general formula (5).
[Chemical Formula 4]
-LCN)ₙ (5)
(in the general formula (5), n represents 1 or 2; L represents an alkenediyl group, an arylenediyl group or a heteroarylenediyl group when n represents 1; and L represents an aklenetriyl group, an arylenetriyl group or a heteroarylenetriyl group when n represents 2)).

7. The photoelectric conversion element according to claim 5, wherein the second compound is a compound represented by the following general formula (6): 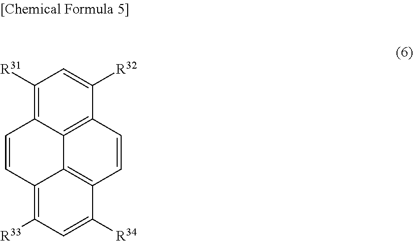
(in the general formula (6), R³¹ to R³⁴ may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, an amino group, a silyl group, —P(═O)R³⁵R³⁶, and a group represented by the following general formula (5); R³⁵ and R³⁶ each represent an aryl group or a heteroaryl group; and at least one of R³¹ to R³⁴ represents a group represented by the following general formula (5):
[Chemical Formula 6]
-LCN)ₙ (5)
(in the general formula (5), n represents 1 or 2; L represents an alkenediyl group, an arylenediyl group or a heteroarylenediyl group when n represents 1; and L represents an aklenetriyl group, an arylenetriyl group or a heteroarylenetriyl group when n represents 2)).

8. The photoelectric conversion element according to claim 5, wherein the second compound is a compound represented by the general formula (7): 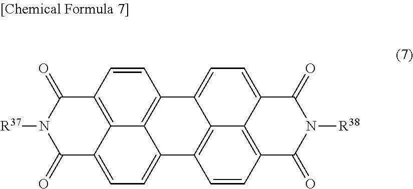
(in the general formula (7), R³⁷ to R³⁸ may be the same or different, and each represent a group selected from the group consisting of hydrogen, an alkyl group, a cycloalkyl group, a heterocyclic group, an alkenyl group, a cycloalkenyl group, an alkynyl group, an alkoxy group, an alkylthio group, an aryl ether group, an aryl thioether group, an aryl group, a heteroaryl group, a halogen, an amino group, a cyano group, a silyl group and —P(═O)R³⁹R⁴⁰; and R³⁹ and R⁴⁰ each represent an aryl group or a heteroaryl group).

9. The photoelectric conversion element according to claim 1, wherein the organic layer includes a photoelectric conversion layer, and the photoelectric conversion layer contains the first compound and the second compound.

10. The photoelectric conversion element according to claim 1, wherein the first compound is a p-type semiconductor material, and the second compound is a n-type semiconductor material.

11. The photoelectric conversion element according to claim 1, wherein the first compound and the second compound each have a charge mobility of 1×10⁻⁹ cm²/Vs or more.

12. The photoelectric conversion element according to claim 1, wherein the organic layer has a thickness of 20 nm or more and 200 nm or less.

13. An image sensor comprising the photoelectric conversion element according to claim 1.

14. The image sensor according to claim 13, including two or more photoelectric conversion elements, at least one of the photoelectric conversion elements being the photoelectric conversion element according to any one of claims 1 to 12.

15. The image sensor according to claim 14, wherein the two or more photoelectric conversion elements have a laminated structure.

16. A solar cell comprising the photoelectric conversion element according to claim 1.

17. A single color detection sensor comprising the photoelectric conversion element according to claim 1.

18. A flexible sensor comprising the photoelectric conversion element according to claim 1.
